(19) United States
(12) Patent Application Publication
(10) Pub. No.: US 2025/0059153 A1
(43) Pub. Date: Feb. 20, 2025

(54) BICYCLIC QUINONES, PHARMACEUTICAL COMPOSITIONS, AND THERAPEUTIC APPLICATIONS

(71) Applicant: IC-MedTech Corp., Las Vegas, NV (US)

(72) Inventors: Stephen M. Coutts, Santa Fe, NM (US); Thomas M. Miller, Las Vegas, NV (US)

(21) Appl. No.: 18/724,480

(22) PCT Filed: Jan. 17, 2023
(86) PCT No.: PCT/US2023/060714

§ 371 Date: Jun. 26, 2024

Related U.S. Application Data
(60) Provisional application No. 63/268,690, filed on Feb. 28, 2022.
(60) Provisional application No. 63/266,867, filed on Jan. 18, 2022.

(51) Int. Cl. C07D 319/16; A61K 31/352; A61K 31/357; A61K 31/4168; A61K 31/541; A61P 33/02; C07D 311/04

(52) U.S. Cl. CPC C07D 319/16; A61K 31/352; A61K 31/357; A61K 31/4168; A61K 31/541; A61P 33/02; C07D 311/04

(57) ABSTRACT

Provided herein are bicyclic quinones, e.g., a compound of Formula (A-I), and pharmaceutical compositions thereof. Also provided herein are methods of their use for treating, preventing, or ameliorating one or more symptoms of a parasite disease.

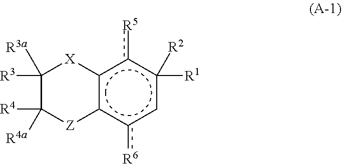

### CROSS REFERENCE TO RELATED APPLICATIONS

This application claims the benefit of the priority of U.S. Provisional Application Nos. 63/266,867, filed Jan. 18, 2022, and 63/268,690, filed Feb. 28, 2022; the disclosure of each of which is incorporated herein by reference in its entirety.

### FIELD

Provided herein are bicyclic quinones and pharmaceutical compositions thereof. Also provided herein are methods of their use for treating, preventing, or ameliorating one or more symptoms of a parasite disease.

### BACKGROUND

Chagas disease is a tropical parasitic disease caused by the protozoan Trypanosoma cruzi, affecting millions of people worldwide. World Health Organization. Chagas disease (also known as American trypanosomiasis), WHO, Geneva, 2021. It is spread mostly by insects known as Triatominae or kissing bugs. Id. Chagas disease is considered a silent pathology since the first symptoms may appear several years after infection. Maguire, N. Engl. J. Med. 2006, 355, 760-761. The symptoms of Chagas disease change over the course of the infection. In the early stage, symptoms are typically either not present or mild, and may include fever, swollen lymph nodes, headaches, or local swelling at the site of the bite. After 8 to 12 weeks, individuals enter the chronic phase of the disease and in 60 to 70%, it never produces further symptoms. The other 30 to 40% of people develop further symptoms 10 to 30 years after the initial infection, including enlargement of the ventricles of the heart in 20 to 30%, leading to heart failure. An enlarged esophagus or an enlarged colon may also occur in 10% of people.

Currently, benznidazole and nifurtimox are only drugs available for treating Chagas disease and both were developed more than four decades ago. Coura et al., Mem. Inst. Oswaldo Cruz. 2002, 97, 3-24. Benznidazole and nifurtimox are also known to have variable efficacy and high toxicity. Id. For example, benznidazole and nifurtimox cause temporary side effects in up to 40% of people, including skin disorders, brain toxicity, and digestive system irritation. Chagas disease (American trypanosomiasis) Fact sheet No. 340. Therefore, there exists a need for an effective therapy for treating Chagas disease.

### SUMMARY OF THE DISCLOSURE

Provided herein is a compound of Formula (A-I):

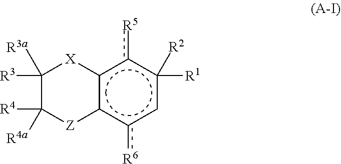

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein:
the symbol “ [symbol] ” represents a single bond or double bond;
X and Z are each independently O or C(R⁷R⁸);
R¹ is absent or —SO₃H;
R² is C₁₋₆ alkyl or C₁₋₆ heteroalkyl;
each R³, R⁴, R⁷, and R⁸ is independently (i) hydrogen, deuterium, cyano, or halo; or (ii) C₁₋₆ alkyl or C₁₋₆ heteroalkyl;
R⁵ and R⁶ are each independently —OH or ═O; and
R^{3a} and R^{4a} are each independently (i) hydrogen, deuterium, cyano, or halo; or (ii) C₁₋₆ alkyl or C₁₋₆ heteroalkyl; or R^{3a} and R^{4a} together with the carbon atoms to which they are attached form a double bond;
wherein each alkyl and heteroalkyl is optionally substituted with one or more, in one embodiment, one, two, three, or four, substituents Q, wherein each Q is independently selected from: (a) deuterium, cyano, halo, imino, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{a})OR^{d}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a};
wherein each Q^{a} is independently selected from: (a) deuterium, cyano, halo, nitro, imino, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{g}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OP(O)(OR^{f})OR^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

Also provided herein is a compound of Formula (A-II):

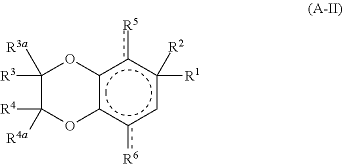

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein:
the symbol “ [symbol] ” represents a single bond or double bond;
R¹ is absent or —SO₃H;
R² is C₁₋₆ alkyl or C₁₋₆ heteroalkyl;
R³ and R⁴ are each independently (i) hydrogen, deuterium, cyano, or halo; or (ii) C₁₋₆ alkyl or C₁₋₆ heteroalkyl;
R⁵ and R⁶ are each independently —OH or ═O; and
R^{3a} and R^{4a} are each independently (i) hydrogen, deuterium, cyano, or halo; or (ii) C₁₋₆ alkyl or C₁₋₆ heteroalkyl; or R^{3a} and R^{4a} together with the carbon atoms to which they are attached form a double bond;
wherein each alkyl and heteroalkyl is optionally substituted with one or more, in one embodiment, one, two, three, or four, substituents Q, wherein each Q is independently selected from: (a) deuterium, cyano, halo, imino, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{al , —C(NR}^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{a})OR^{d}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a};
wherein each Q^{a} is independently selected from: (a) deuterium, cyano, halo, nitro, imino, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OP(O)(OR^{f})OR^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NRS(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

Additionally provided herein is a pharmaceutical composition comprising a compound of Formula (A-I), (A-II), or (I), or an enantiomer, a mixture of enantiomers, or a mixture of diastereomers thereof, or a pharmaceutically acceptable salt, solvate, hydrate, or prodrug thereof; and a pharmaceutically acceptable excipient.

Furthermore, a method of treating, preventing, or alleviating one or more symptoms of a parasitic disease in a subject, comprising administering to the subject in need thereof a therapeutically effective amount of a compound of Formula (A-I), (A-II), or (I), or an enantiomer, a mixture of enantiomers, or a mixture of diastereomers thereof, or a pharmaceutically acceptable salt, solvate, hydrate, or prodrug thereof.

Provided herein is a method of inhibiting the growth of a parasite, comprising contacting the parasite with an effective amount of a compound of Formula (A-I), (A-II), or (I), or an enantiomer, a mixture of enantiomers, or a mixture of diastereomers thereof, or a pharmaceutically acceptable salt, solvate, hydrate, or prodrug thereof.

### DETAILED DESCRIPTION

To facilitate understanding of the disclosure set forth herein, a number of terms are defined below.

Generally, the nomenclature used herein and the laboratory procedures in organic chemistry, medicinal chemistry, biochemistry, biology, and pharmacology described herein are those well-known and commonly employed in the art. Unless defined otherwise, all technical and scientific terms used herein generally have the same meaning as commonly understood by one of ordinary skill in the art to which this disclosure belongs.

The term “subject” refers to an animal, including, but not limited to, a primate (e.g., human), livestock, a domestic pet, cow, pig, sheep, goat, horse, dog, cat, rabbit, rat, or mouse. The terms “subject” and “patient” are used interchangeably herein in reference, for example, to a mammalian subject, such as a human subject, in one embodiment, a human.

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself.

The terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition.

The terms “alleviate” and “alleviating” refer to easing or reducing one or more symptoms (e.g., pain) of a disorder, disease, or condition. The terms can also refer to reducing adverse effects associated with an active ingredient. Sometimes, the beneficial effects that a subject derives from a prophylactic or therapeutic agent do not result in a cure of the disorder, disease, or condition.

The term “contacting” or “contact” is meant to refer to bringing together of a therapeutic agent and a biological molecule (e.g., a protein, enzyme, RNA, or DNA), cell, or tissue such that a physiological and/or chemical effect takes place as a result of such contact. Contacting can take place in vitro, ex vivo, or in vivo. In one embodiment, a therapeutic agent is contacted with a biological molecule in vitro to determine the effect of the therapeutic agent on the biological molecule. In another embodiment, a therapeutic agent is contacted with a cell in cell culture (in vitro) to determine the effect of the therapeutic agent on the cell. In yet another embodiment, the contacting of a therapeutic agent with a biological molecule, cell, or tissue includes the administration of a therapeutic agent to a subject having the biological molecule, cell, or tissue to be contacted.

The term “therapeutically effective amount” or “effective amount” is meant to include the amount of a compound that, when administered, is sufficient to prevent development of, or alleviate to some extent, one or more of the symptoms of the disorder, disease, or condition being treated. The term “therapeutically effective amount” or “effective amount” also refers to the amount of a compound that is sufficient to elicit a biological or medical response of a biological molecule (e.g., a protein, enzyme, RNA, or DNA), cell, tissue, system, animal, or human, which is being sought by a researcher, veterinarian, medical doctor, or clinician.

The term “IC₅₀” or “EC₅₀” refers to an amount, concentration, or dosage of a compound that is required for 50% inhibition of a maximal response in an assay that measures such a response.

The term “pharmaceutically acceptable carrier,” “pharmaceutically acceptable excipient,” “physiologically acceptable carrier,” or “physiologically acceptable excipient” refers to a pharmaceutically acceptable material, composition, or vehicle, such as a liquid or solid filler, diluent, solvent, or encapsulating material. In one embodiment, each component is “pharmaceutically acceptable” in the sense of being compatible with the other ingredients of a pharmaceutical formulation, and suitable for use in contact with the tissue or organ of a subject (e.g., a human) without excessive toxicity, irritation, allergic response, immunogenicity, or other problems or complications, and commensurate with a reasonable benefit/risk ratio. See, e.g., Remington: The Science and Practice of Pharmacy, 23rd ed.; Adejare Ed.; Academic Press, 2020; Handbook of Pharmaceutical Excipients, 9th ed.; Sheskey et al., Eds.; Pharmaceutical Press, 2020; Handbook of Pharmaceutical Additives, 3rd ed.; Ash and Ash Eds.; Synapse Information Resources, 2007; Pharmaceutical Preformulation and Formulation, 2nd ed.; Gibson Ed.; CRC Press, 2009.

The term “about” or “approximately” means an acceptable error for a particular value as determined by one of ordinary skill in the art, which depends in part on how the value is measured or determined. In certain embodiments, the term “about” or “approximately” means within 1, 2, or 3 standard deviations. In certain embodiments, the term “about” or “approximately” means within 25%, 20%, 15%, 10%, 9%, 8%, 7%, 6%, 5%, 4%, 3%, 2%, 1%, 0.5%, or 0.05% of a given value or range.

The term “alkyl” refers to a linear or branched saturated monovalent hydrocarbon radical, wherein the alkyl is optionally substituted with one or more substituents Q as described herein. For example, C₁₋₆ alkyl refers to a linear saturated monovalent hydrocarbon radical of 1 to 6 carbon atoms or a branched saturated monovalent hydrocarbon radical of 3 to 6 carbon atoms. In certain embodiments, the alkyl is a linear saturated monovalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ alkyl groups are also referred as “lower alkyl.” Examples of alkyl groups include, but are not limited to, methyl, ethyl, propyl (including all isomeric forms, e.g., n-propyl and isopropyl), butyl (including all isomeric forms, e.g., n-butyl, isobutyl, sec-butyl, and t-butyl), pentyl (including all isomeric forms, e.g., n-pentyl, isopentyl, sec-pentyl, neopentyl, and tert-pentyl), and hexyl (including all isomeric forms, e.g., n-hexyl, isohexyl, and sec-hexyl).

The term “heteroalkyl” refers to a linear or branched saturated monovalent hydrocarbon radical that contains one or more heteroatoms on its main chain, each independently selected from O, S, and N. The heteroalkyl is optionally substituted with one or more substituents Q as described herein. For example, C₁₋₆ heteroalkyl refers to a linear saturated monovalent hydrocarbon radical of 1 to 6 carbon atoms or a branched saturated monovalent hydrocarbon radical of 3 to 6 carbon atoms. In certain embodiments, the heteroalkyl is a linear saturated monovalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ heteroalkyl groups are also referred as “lower heteroalkyl.” Examples of heteroalkyl groups include, but are not limited to, —OCH₃, —OCH₂CH₃, —CH₂OCH₃, —NHCH₃, —ONHCH₃, —NHOCH₃, —SCH₃, —CH₂NHCH₂CH₃, and —NHCH₂CH₂CH₃. Examples of substituted heteroalkyl groups include, but are not limited to, —CH₂NHC(O)CH₃ and —NHC(O)CH₂CH₃.

The term “alkenyl” refers to a linear or branched monovalent hydrocarbon radical, which contains one or more, in one embodiment, one, two, three, or four, in another embodiment, one, carbon-carbon double bond(s). The alkenyl is optionally substituted with one or more substituents Q as described herein. The term “alkenyl” embraces radicals having a “cis” or “trans” configuration or a mixture thereof, or alternatively, a “Z” or “E” configuration or a mixture thereof, as appreciated by those of ordinary skill in the art. For example, C₂-6 alkenyl refers to a linear unsaturated monovalent hydrocarbon radical of 2 to 6 carbon atoms or a branched unsaturated monovalent hydrocarbon radical of 3 to 6 carbon atoms. In certain embodiments, the alkenyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of alkenyl groups include, but are not limited to, ethenyl, propenyl (including all isomeric forms, e.g., propen-1-yl, propen-2-yl, and allyl), and butenyl (including all isomeric forms, e.g., buten-1-yl, buten-2-yl, buten-3-yl, and 2-buten-1-yl).

The term “alkynyl” refers to a linear or branched monovalent hydrocarbon radical, which contains one or more, in one embodiment, one, two, three, or four, in another embodiment, one, carbon-carbon triple bond(s). An alkynyl group does not contain a carbon-carbon double bond. The alkynyl is optionally substituted with one or more substituents Q as described herein. For example, C₂-6 alkynyl refers to a linear unsaturated monovalent hydrocarbon radical of 2 to 6 carbon atoms or a branched unsaturated monovalent hydrocarbon radical of 4 to 6 carbon atoms. In certain embodiments, the alkynyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 4 to 20 (C₄₋₂₀), 4 to 15 (C₄₋₁₅), 4 to 10 (C₄₋₁₀), or 4 to 6 (C₄₋₆) carbon atoms. Examples of alkynyl groups include, but are not limited to, ethynyl (—C≡CH), propynyl (including all isomeric forms, e.g., 1-propynyl (—C≡CCH₃) and propargyl (—CH₂C≡CH)), butynyl (including all isomeric forms, e.g., 1-butyn-1-yl and 2-butyn-1-yl), pentynyl (including all isomeric forms, e.g., 1-pentyn-1-yl and 1-methyl-2-butyn-1-yl), and hexynyl (including all isomeric forms, e.g., 1-hexyn-1-yl and 2-hexyn-1-yl).

The term “cycloalkyl” refers to a cyclic monovalent hydrocarbon radical, which is optionally substituted with one or more substituents Q as described herein. In one embodiment, the cycloalkyl is a saturated or unsaturated but non-aromatic, and/or bridged or non-bridged, and/or fused bicyclic group. In certain embodiments, the cycloalkyl has from 3 to 20 (C₃₋₂₀), from 3 to 15 (C₃₋₁₅), from 3 to 10 (C₃₋₁₀), or from 3 to 7 (C₃₋₇) carbon atoms. In one embodiment, the cycloalkyl is monocyclic. In another embodiment, the cycloalkyl is bicyclic. In yet another embodiment, the cycloalkyl is tricyclic. In still another embodiment, the cycloalkyl is polycyclic. Examples of cycloalkyl groups include, but are not limited to, cyclopropyl, cyclobutyl, cyclopentyl, cyclopentenyl, cyclohexyl, cyclohexenyl, cyclohexadienyl, cycloheptyl, cycloheptenyl, bicyclo[1.1.1]pentyl, bicyclo[2.1.1]hexyl, bicyclo[2.2.1]heptyl, bicyclo[2.2.2]octyl, decalinyl, and adamantyl.

The term “aryl” refers to a monovalent monocyclic aromatic hydrocarbon radical and/or monovalent polycyclic aromatic hydrocarbon radical that contain at least one aromatic carbon ring. In certain embodiments, the aryl has from 6 to 20 (C₆₋₂₀), from 6 to 15 (C₆₋₁₅), or from 6 to 10 (C₆₋₁₀) ring carbon atoms. Examples of aryl groups include, but are not limited to, phenyl, naphthyl, fluorenyl, azulenyl, anthryl, phenanthryl, pyrenyl, biphenyl, and terphenyl. The aryl also refers to bicyclic or tricyclic carbon rings, where one of the rings is aromatic and the others of which may be saturated, partially unsaturated, or aromatic, for example, dihydronaphthyl, indenyl, indanyl, or tetrahydronaphthyl (tetralinyl). In one embodiment, the aryl is monocyclic. In another embodiment, the aryl is bicyclic. In yet another embodiment, the aryl is tricyclic. In still another embodiment, the aryl is polycyclic. In certain embodiments, the aryl is optionally substituted with one or more substituents Q as described herein.

The term “aralkyl” or “arylalkyl” refers to a monovalent alkyl group substituted with one or more aryl groups. In certain embodiments, the aralkyl has from 7 to 30 (C₇₋₃₀), from 7 to 20 (C₇₋₂₀), or from 7 to 16 (C₇₋₁₆) carbon atoms. Examples of aralkyl groups include, but are not limited to, benzyl, phenylethyl (including all isomeric forms, e.g., 1-phenylethyl and 2-phenylethyl), and phenylpropyl (including all isomeric forms, e.g., 1-phenylpropyl, 2-phenylpropyl, and 3-phenylpropyl). In certain embodiments, the aralkyl is optionally substituted with one or more substituents Q as described herein.

The term “heteroaryl” refers to a monovalent monocyclic aromatic group or monovalent polycyclic aromatic group that contain at least one aromatic ring, wherein at least one aromatic ring contains one or more heteroatoms, each independently selected from O, S, and N, in the ring. For a heteroaryl group containing a heteroaromatic ring and a nonaromatic heterocyclic ring, the heteroaryl group is not bonded to the rest of a molecule through its nonaromatic heterocyclic ring. Each ring of a heteroaryl group can contain one or two O atoms, one or two S atoms, and/or one to four N atoms; provided that the total number of heteroatoms in each ring is four or less and each ring contains at least one carbon atom. In certain embodiments, the heteroaryl has from 5 to 20, from 5 to 15, or from 5 to 10 ring atoms. In one embodiment, the heteroaryl is monocyclic. Examples of monocyclic heteroaryl groups include, but are not limited to, furanyl, imidazolyl, isothiazolyl, isoxazolyl, oxadiazolyl, oxazolyl, pyrazinyl, pyrazolyl, pyridazinyl, pyridyl, pyrimidinyl, pyrrolyl, thiadiazolyl, thiazolyl, thienyl, tetrazolyl, triazinyl, and triazolyl. In another embodiment, the heteroaryl is bicyclic. Examples of bicyclic heteroaryl groups include, but are not limited to, benzofuranyl, benzimidazolyl, benzoisoxazolyl, benzopyranyl, benzothiadiazolyl, benzothiazolyl, benzothienyl, benzotriazolyl, benzoxazolyl, furopyrindyl (including all isomeric forms, e.g., furo[2,3-b]pyridinyl, furo[2,3-c]pyridinyl, furo[3,2-b]pyridinyl, furo[3,2-c]pyridinyl, furo[3,4-b]pyridinyl, and furo[3,4-c]pyridinyl), imidazopyridinyl (including all isomeric forms, e.g., imidazo[1,2-a]pyridinyl, imidazo[4,5-b]-pyridinyl, and imidazo[4,5-c]pyridinyl), imidazothiazolyl (including all isomeric forms, e.g., imidazo[2,1-b]thiazolyl and imidazo[4,5-d]thiazolyl), indazolyl, indolizinyl, indolyl, isobenzo-furanyl, isobenzothienyl (i.e., benzo[c]thienyl), isoindolyl, isoquinolinyl, naphthyridinyl (including all isomeric forms, e.g., 1,5-naphthyridinyl, 1,6-naphthyridinyl, 1,7-naphthyridinyl, and 1,8-naphthyridinyl), oxazolopyridinyl (including all isomeric forms, e.g., oxazolo[4,5-b]-pyridinyl, oxazolo[4,5-c]pyridinyl, oxazolo[5,4-b]pyridinyl, and oxazolo[5,4-c]pyridinyl), phthalazinyl, pteridinyl, purinyl, pyrrolopyridyl (including all isomeric forms, e.g., pyrrolo[2,3-b]pyridinyl, pyrrolo[2,3-c]pyridinyl, pyrrolo[3,2-b]pyridinyl, and pyrrolo[3,2-c]pyridinyl), quinolinyl, quinoxalinyl, quinazolinyl, thiadiazolopyrimidyl (including all isomeric forms, e.g., [1,2,5]thiadiazolo[3,4-d]pyrimidinyl and [1,2,3]thiadiazolo[4,5-d]pyrimidinyl), and thieno-pyridyl (including all isomeric forms, e.g., thieno[2,3-b]pyridinyl, thieno[2,3-c]pyridinyl, thieno-[3,2-b]pyridinyl, and thieno[3,2-c]pyridinyl). In yet another embodiment, the heteroaryl is tricyclic. Examples of tricyclic heteroaryl groups include, but are not limited to, acridinyl, benzindolyl, carbazolyl, dibenzofuranyl, perimidinyl, phenanthridinyl, phenanthrolinyl (including all isomeric forms, e.g., 1,5-phenanthrolinyl, 1,6-phenanthrolinyl, 1,7-phen-anthrolinyl, 1,9-phenanthrolinyl, and 2,10-phenanthrolinyl), phenarsazinyl, phenazinyl, pheno-thiazinyl, phenoxazinyl, and xanthenyl. In certain embodiments, the heteroaryl is optionally substituted with one or more substituents Q as described herein.

The term “heterocyclyl” or “heterocyclic” refers to a monovalent monocyclic non-aromatic ring system or monovalent polycyclic ring system that contains at least one non-aromatic ring, wherein one or more of the non-aromatic ring atoms are heteroatoms, each independently selected from O, S, and N; and the remaining ring atoms are carbon atoms. For a heterocyclyl group containing a heteroaromatic ring and a nonaromatic heterocyclic ring, the heterocyclyl group is not bonded to the rest of a molecule through the heteroaromatic ring. In certain embodiments, the heterocyclyl or heterocyclic group has from 3 to 20, from 3 to 15, from 3 to 10, from 3 to 8, from 4 to 7, or from 5 to 6 ring atoms. In certain embodiments, the heterocyclyl is a monocyclic, bicyclic, tricyclic, or tetracyclic ring system, which may be fused or bridged, and in which nitrogen or sulfur atoms may be optionally oxidized, nitrogen atoms may be optionally quaternized, and some rings may be partially or fully saturated, or aromatic. The heterocyclyl may be attached to the main structure at any heteroatom or carbon atom which results in the creation of a stable compound. Examples of heterocyclyls and heterocyclic groups include, but are not limited to, azepinyl, benzodioxanyl, benzodioxolyl, benzofuranonyl, chromanyl, decahydroisoquinolinyl, dihydrobenzofuranyl, dihydrobenzisothiazolyl, dihydro-benzisoxazinyl (including all isomeric forms, e.g., 1,4-dihydrobenzo[d][1,3]oxazinyl, 3,4-dihydrobenzo[c][1,2]-oxazinyl, and 3,4-dihydrobenzo[d][1,2]oxazinyl), dihydrobenzothienyl, dihydroisobenzofuranyl, dihydrobenzo[c]thienyl, dihydrofuryl, dihydroisoindolyl, dihydro-pyranyl, dihydropyrazolyl, dihydropyrazinyl, dihydropyridinyl, dihydropyrimidinyl, dihydro-pyrrolyl, dioxolanyl, 1,4-dithianyl, furanonyl, imidazolidinyl, imidazolinyl, indolinyl, isochromanyl, isoindolinyl, isothiazolidinyl, isoxazolidinyl, morpholinyl, octahydroindolyl, octahydroisoindolyl, oxazolidinonyl, oxazolidinyl, oxiranyl, piperazinyl, piperidinyl, 4-piperidonyl, pyrazolidinyl, pyrazolinyl, pyrrolidinyl, pyrrolinyl, quinuclidinyl, tetrahydrofuryl, tetrahydroisoquinolinyl, tetrahydropyranyl, tetrahydrothienyl, thiamorpholinyl, thiazolidinyl, thiochromanyl, tetrahydroquinolinyl, and 1,3,5-trithianyl. In certain embodiments, the heterocyclyl is optionally substituted with one or more substituents Q as described herein.

The term “halogen,” “halide,” or “halo” refers to fluoro, chloro, bromo, and/or iodo.

The term “optionally substituted” is intended to mean that a group or substituent, such as an alkyl, heteroalkyl, alkenyl, alkynyl, cycloalkyl, aryl, aralkyl, heteroaryl, or heterocyclyl group, may be substituted with one or more, in one embodiment, one, two, three, or four, substituents Q, each of which is independently selected from, e.g., (a) deuterium (-D), cyano (—CN), halo, imino (=NH), nitro (—NO₂), and oxo (=O); (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{a})OR^{d}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR R, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl optionally substituted with one or more, in one embodiment, one, two, three, or four, substituents Q^{a}. As used herein, all groups that can be substituted are “optionally substituted.”

In one embodiment, each Q^{a} is independently selected from: (a) deuterium, cyano, halo, imino, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OP(O)(OR^{f})OR^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In certain embodiments, “optically active” and “enantiomerically active” refer to a collection of molecules, which has an enantiomeric excess of no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, or no less than about 99.8%. In certain embodiments, an optically active compound comprises about 95% or more of one enantiomer and about 5% or less of the other enantiomer based on the total weight of the enantiomeric mixture in question. In certain embodiments, an optically active compound comprises about 98% or more of one enantiomer and about 2% or less of the other enantiomer based on the total weight of the enantiomeric mixture in question. In certain embodiments, an optically active compound comprises about 99% or more of one enantiomer and about 1% or less of the other enantiomer based on the total weight of the enantiomeric mixture in question.

In describing an optically active compound, the prefixes R and S are used to denote the absolute configuration of the compound about its chiral center(s). The (+) and (−) are used to denote the optical rotation of the compound, that is, the direction in which a plane of polarized light is rotated by the optically active compound. The (−) prefix indicates that the compound is levorotatory, that is, the compound rotates the plane of polarized light to the left or counterclockwise. The (+) prefix indicates that the compound is dextrorotatory, that is, the compound rotates the plane of polarized light to the right or clockwise. However, the sign of optical rotation, (+) and (−), is not related to the absolute configuration of the compound, R and S.

The term “isotopically enriched” refers to a compound that contains an unnatural proportion of an isotope at one or more of the atoms that constitute such a compound. In certain embodiments, an isotopically enriched compound contains unnatural proportions of one or more isotopes, including, but not limited to, hydrogen (¹H), deuterium (²H), tritium (³H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine-123 (¹²³I), iodine-125 (¹²⁵I), iodine-127 (¹²⁷I), iodine-129 (¹²⁹I), and iodine-131 (¹³¹I). In certain embodiments, an isotopically enriched compound is in a stable form, that is, non-radioactive. In certain embodiments, an isotopically enriched compound contains unnatural proportions of one or more isotopes, including, but not limited to, hydrogen (¹H), deuterium (²H), carbon-12 (¹²C), carbon-13 (¹³C), nitrogen-14 (¹⁴N), nitrogen-15 (¹⁵N), oxygen-16 (¹⁶O), oxygen-17 (¹⁷O), oxygen-18 (¹⁸O), fluorine-17 (¹⁷F), phosphorus-31 (³¹P), sulfur-32 (³²S), sulfur-33 (³³S), sulfur-34 (³⁴S), sulfur-36 (³⁶S), chlorine-35 (³⁵Cl), chlorine-37 (³⁷Cl), bromine-79 (⁷⁹Br), bromine-81 (⁸¹Br), and iodine-127 (¹²⁷I). In certain embodiments, an isotopically enriched compound is in an unstable form, that is, radioactive. In certain embodiments, an isotopically enriched compound contains unnatural proportions of one or more isotopes, including, but not limited to, tritium (3H), carbon-11 (¹¹C), carbon-14 (¹⁴C), nitrogen-13 (¹³N), oxygen-14 (¹⁴O), oxygen-15 (¹⁵O), fluorine-18 (¹⁸F), phosphorus-32 (³²P), phosphorus-33 (³³P), sulfur-35 (³⁵S), chlorine-36 (³⁶Cl), iodine-123 (¹²³I), iodine-125 (¹²⁵I), iodine-129 (¹²⁹I), and iodine-131 (¹³¹I). It will be understood that, in a compound as provided herein, any hydrogen can be ²H, as example, or any carbon can be ¹³C, as example, or any nitrogen can be ¹⁵N, as example, or any oxygen can be ¹⁸O, as example, where feasible according to the judgment of one of ordinary skill in the art.

The term “isotopic enrichment” refers to the percentage of incorporation of a less prevalent isotope (e.g., D for deuterium or hydrogen-2) of an element at a given position in a molecule in the place of a more prevalent isotope (e.g., ¹H for protium or hydrogen-1) of the element. As used herein, when an atom at a particular position in a molecule is designated as a particular less prevalent isotope, it is understood that the abundance of that isotope at that position is substantially greater than its natural abundance.

The term “isotopic enrichment factor” refers the ratio between the isotopic abundance in an isotopically enriched compound and the natural abundance of a specific isotope.

The term “hydrogen” or the symbol “H” refers to the composition of naturally occurring hydrogen isotopes, which include protium (H), deuterium (²H or D), and tritium (H), in their natural abundances. Protium is the most common hydrogen isotope having a natural abundance of more than 99.98%. Deuterium is a less prevalent hydrogen isotope having a natural abundance of about 0.0156%.

The term “deuterium enrichment” refers to the percentage of incorporation of deuterium at a given position in a molecule in the place of hydrogen. For example, deuterium enrichment of 1% at a given position means that 1% of molecules in a given sample contain deuterium at the specified position. Because the naturally occurring distribution of deuterium is about 0.0156% on average, deuterium enrichment at any position in a compound synthesized using non-enriched starting materials is about 0.0156% on average. As used herein, when a particular position in an isotopically enriched compound is designated as having deuterium, it is understood that the abundance of deuterium at that position in the compound is substantially greater than its natural abundance (0.0156%).

The term “carbon” or the symbol “C” refers to the composition of naturally occurring carbon isotopes, which include carbon-12 (¹²C) and carbon-13 (¹³C) in their natural abundances. Carbon-12 is the most common carbon isotope having a natural abundance of more than 98.89%. Carbon-13 is a less prevalent carbon isotope having a natural abundance of about 1.11%.

The term “carbon-13 enrichment” or “¹³C enrichment” refers to the percentage of incorporation of carbon-13 at a given position in a molecule in the place of carbon. For example, carbon-13 enrichment of 10% at a given position means that 10% of molecules in a given sample contain carbon-13 at the specified position. Because the naturally occurring distribution of carbon-13 is about 1.11% on average, carbon-13 enrichment at any position in a compound synthesized using non-enriched starting materials is about 1.11% on average. As used herein, when a particular position in an isotopically enriched compound is designated as having carbon-13, it is understood that the abundance of carbon-13 at that position in the compound is substantially greater than its natural abundance (1.11%).

The terms “substantially pure” and “substantially homogeneous” mean, when referred to a substance, sufficiently homogeneous to appear free of readily detectable impurities as determined by a standard analytical method used by one of ordinary skill in the art, including, but not limited to, thin layer chromatography (TLC), gel electrophoresis, high performance liquid chromatography (HPLC), gas chromatography (GC), nuclear magnetic resonance (NMR), and mass spectrometry (MS); or sufficiently pure such that further purification would not detectably alter the physical, chemical, biological, and/or pharmacological properties, such as enzymatic and biological activities, of the substance. In certain embodiments, “substantially pure” or “substantially homogeneous” refers to a collection of molecules, wherein at least about 95%, at least about 96%, at least about 97%, at least about 98%, at least about 99%, or at least about 99.5% by weight of the molecules are a single compound, including a single enantiomer, a racemic mixture, or a mixture of enantiomers, as determined by standard analytical methods. As used herein, when an atom at a particular position in an isotopically enriched molecule is designated as a particular less prevalent isotope, a molecule that contains other than the designated isotope at the specified position is an impurity with respect to the isotopically enriched compound. Thus, for a deuterated compound that has an atom at a particular position designated as deuterium, a compound that contains a protium at the same position is an impurity.

The term “solvate” refers to a complex or aggregate formed by one or more molecules of a solute, e.g., a compound provided herein, and one or more molecules of a solvent, which are present in stoichiometric or non-stoichiometric amount. Suitable solvents include, but are not limited to, water, methanol, ethanol, n-propanol, isopropanol, and acetic acid. In certain embodiments, the solvent is pharmaceutically acceptable. In one embodiment, the complex or aggregate is in a crystalline form. In another embodiment, the complex or aggregate is in a noncrystalline form. Where the solvent is water, the solvate is a hydrate. Examples of hydrates include, but are not limited to, a hemihydrate, monohydrate, dihydrate, trihydrate, tetrahydrate, and pentahydrate.

The phrase “an enantiomer, a mixture of enantiomers, a mixture of two or more diastereomers, a tautomer, a mixture of two or more tautomers, or an isotopic variant thereof; or a pharmaceutically acceptable salt, solvate, hydrate, or prodrug thereof” has the same meaning as the phrase “(i) an enantiomer, a mixture of enantiomers, a mixture of two or more diastereomers, a tautomer, a mixture of two or more tautomers, or an isotopic variant of the compound referenced therein; (ii) a pharmaceutically acceptable salt, solvate, hydrate, or prodrug of the compound referenced therein; or (iii) a pharmaceutically acceptable salt, solvate, hydrate, or prodrug of an enantiomer, a mixture of enantiomers, a mixture of two or more diastereomers, a tautomer, a mixture of two or more tautomers, or an isotopic variant of the compound referenced therein.”

### Compounds

In one embodiment, provided herein is a compound of Formula (A-I):

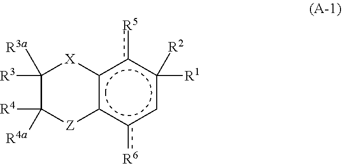

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein:
the symbol “ [symbol] ” represents a single bond or double bond;
X and Z are each independently O or C(R⁷R⁸);
R¹ is absent or —SO₃H;
R² is C₁₋₆ alkyl or C₁₋₆ heteroalkyl;
each R³, R⁴, R⁷, and R⁸ is independently (i) hydrogen, deuterium, cyano, or halo; or (ii) C₁₋₆ alkyl or C₁₋₆ heteroalkyl;
R⁵ and R⁶ are each independently —OH or ═O; and
R^{3a} and R^{4a} are each independently (i) hydrogen, deuterium, cyano, or halo; or (ii) C₁₋₆ alkyl or C₁₋₆ heteroalkyl; or R^{3a} and R^{4a} together with the carbon atoms to which they are attached form a double bond;
wherein each alkyl and heteroalkyl is optionally substituted with one or more, in one embodiment, one, two, three, or four, substituents Q, wherein each Q is independently selected from: (a) deuterium, cyano, halo, imino, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{a})OR^{d}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a};
wherein each Q^{a} is independently selected from: (a) deuterium, cyano, halo, nitro, imino, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OP(O)(OR^{f})OR^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In certain embodiments, in Formula (A-I), X is O. In certain embodiments, in Formula (A-I), X is C(R⁷R⁸), wherein R⁷ and R⁸ are each as defined herein. In certain embodiments, in Formula (A-I), X is C(H₂).

In certain embodiments, in Formula (A-I), Z is O. In certain embodiments, in Formula (A-I), Z is C(R⁷R⁸), wherein R⁷ and R⁸ are each as defined herein. In certain embodiments, in Formula (A-I), Z is C(H₂).

In certain embodiments, in Formula (A-I), X and/or Y are O. In certain embodiments, in Formula (A-I), X and Z are each O. In certain embodiments, in Formula (A-I), X or Y is O. In certain embodiments, in Formula (A-I), X is O and Z is C(R⁷R⁸), wherein R⁷ and R⁸ are each as defined herein. In certain embodiments, in Formula (A-I), X is O and Z is C(H₂). In certain embodiments, in Formula (A-I), X is C(R⁷R⁸) and Z is 0, wherein R⁷ and R⁸ are each as defined herein. In certain embodiments, in Formula (A-I), X is C(H₂) and Z is O.

In certain embodiments, in Formula (A-I), R⁷ is hydrogen. In certain embodiments, in Formula (A-I), R⁷ is deuterium. In certain embodiments, in Formula (A-I), R⁷ is cyano. In certain embodiments, in Formula (A-I), R⁷ is halo. In certain embodiments, in Formula (A-I), R⁷ is fluoro, chloro, or bromo. In certain embodiments, in Formula (A-I), R⁷ is fluoro. In certain embodiments, in Formula (A-I), R⁷ is chloro. In certain embodiments, in Formula (A-I), R⁷ is C₁₋₆ alkyl, optionally substituted with one or more substituents Q. In certain embodiments, in Formula (A-I), R⁷ is methyl. In certain embodiments, in Formula (A-I), R⁷ is C₁₋₆ heteroalkyl, optionally substituted with one or more substituents Q. In certain embodiments, in Formula (A-I), R⁷ is trifluoromethyl.

In certain embodiments, in Formula (A-I), R⁸ is hydrogen. In certain embodiments, in Formula (A-I), R⁸ is deuterium. In certain embodiments, in Formula (A-I), R⁸ is cyano. In certain embodiments, in Formula (A-I), R⁸ is halo. In certain embodiments, in Formula (A-I), R⁸ is fluoro, chloro, or bromo. In certain embodiments, in Formula (A-I), R⁸ is fluoro. In certain embodiments, in Formula (A-I), R⁸ is chloro. In certain embodiments, in Formula (A-I), R⁸ is C₁₋₆ alkyl, optionally substituted with one or more substituents Q. In certain embodiments, in Formula (A-I), R⁸ is methyl. In certain embodiments, in Formula (A-I), R⁸ is C₁₋₆ heteroalkyl, optionally substituted with one or more substituents Q. In certain embodiments, in Formula (A-I), R⁸ is trifluoromethyl.

In another embodiment, provided herein is a compound of Formula (A-II):

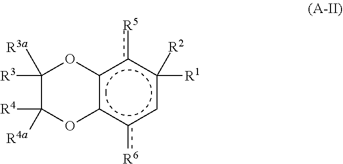

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein:
the symbol “ [symbol] ” represents a single bond or double bond;
R¹ is absent or —SO₃H;
R² is C₁₋₆ alkyl or C₁₋₆ heteroalkyl;
R³ and R⁴ are each independently (i) hydrogen, deuterium, cyano, or halo; or (ii) C₁₋₆ alkyl or C₁₋₆ heteroalkyl;
R⁵ and R⁶ are each independently —OH or ═O; and
R^{3a} and R^{4a} are each independently (i) hydrogen, deuterium, cyano, or halo; or (ii) C₁₋₆ alkyl or C₁₋₆ heteroalkyl; or R^{3a} and R^{4a} together with the carbon atoms to which they are attached form a double bond;
wherein each alkyl and heteroalkyl is optionally substituted with one or more, in one embodiment, one, two, three, or four, substituents Q, wherein each Q is independently selected from: (a) deuterium, cyano, halo, imino, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{a})OR^{d}, —OS(O)R^{a}, —OS(O)₂R^{a}, —OS(O)NR R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a};
wherein each Q^{a} is independently selected from: (a) deuterium, cyano, halo, nitro, imino, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OP(O)(OR^{f})OR^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In one embodiment, provided herein is a compound of Formula (I):

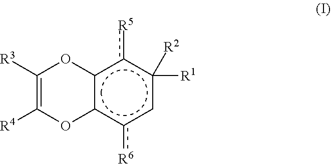

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein:
the symbol “ [symbol] ” represents a single bond or double bond;
R¹ is absent or —SO₃H;
R² is C₁₋₆ alkyl or C₁₋₆ heteroalkyl;
R³ and R⁴ are each independently (i) hydrogen, deuterium, cyano, or halo; or (ii) C₁₋₆ alkyl or C₁₋₆ heteroalkyl; and
R⁵ and R⁶ are each independently —OH or ═O;
wherein each alkyl and heteroalkyl is optionally substituted with one or more, in one embodiment, one, two, three, or four, substituents Q, wherein each Q is independently selected from: (a) deuterium, cyano, halo, imino, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{a})OR^{d}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a};
wherein each Q^{a} is independently selected from: (a) deuterium, cyano, halo, nitro, imino, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}—C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OP(O)(OR^{f})OR^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In another embodiment, provided herein is a compound of Formula (II):

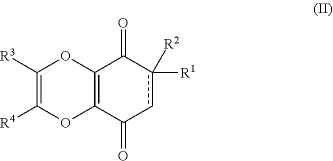

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein:
the symbol “ [symbol] ” represents a carbon-carbon single bond or double bond;
R¹ is (i) absent when the symbol “ [symbol] ” represents a carbon-carbon double bond or (ii) —SO₃H when the symbol “ [symbol] ” represents a carbon-carbon double bond; and
R², R³, and R⁴ are each as defined herein.

In yet another embodiment, provided herein is a compound of Formula (III):

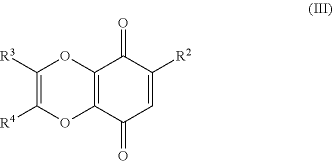

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R², R³, and R⁴ are each as defined herein.

In yet another embodiment, provided herein is a compound of Formula (IV):

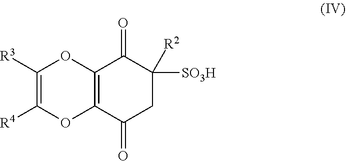

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R², R³, and R⁴ are each as defined herein.

In still another embodiment, provided herein is a compound of Formula (V):

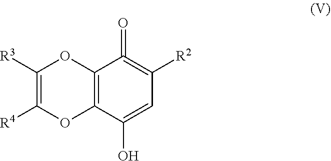

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R², R³, and R⁴ are each as defined herein.

In certain embodiments, in any one of Formulae (I) to (V), R² is C₁₋₆ alkyl, optionally substituted with one or more substituents Q. In certain embodiments, in any one of Formulae (I) to (V), R² is methyl, ethyl, or propyl. In certain embodiments, in any one of Formulae (I) to (V), R² is methyl. In certain embodiments, in any one of Formulae (I) to (V), R² is C₁₋₆ heteroalkyl, optionally substituted with one or more substituents Q.

In one embodiment, provided herein is a compound of Formula (VI):

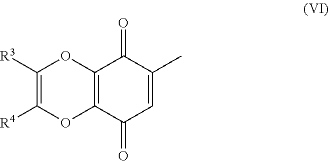

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R³ and R⁴ are each as defined herein.

In another embodiment, provided herein is a compound of Formula (VII):

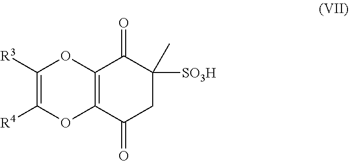

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R³ and R⁴ are each as defined herein.

In yet another embodiment, provided herein is a compound of Formula (VIII):

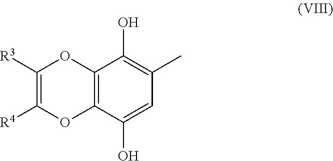

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R³ and R⁴ are each as defined herein.

In certain embodiments, in any one of Formulae (I) to (VIII), R³ is hydrogen. In certain embodiments, in any one of Formulae (I) to (VIII), R³ is deuterium. In certain embodiments, in any one of Formulae (I) to (VIII), R³ is cyano. In certain embodiments, in any one of Formulae (I) to (VIII), R³ is halo. In certain embodiments, in any one of Formulae (I) to (VIII), R³ is fluoro, chloro, or bromo. In certain embodiments, in any one of Formulae (I) to (VIII), R³ is fluoro. In certain embodiments, in any one of Formulae (I) to (VIII), R³ is chloro. In certain embodiments, in any one of Formulae (I) to (VIII), R³ is C₁₋₆ alkyl, optionally substituted with one or more substituents Q. In certain embodiments, in any one of Formulae (I) to (VIII), R³ is methyl, ethyl, or propyl. In certain embodiments, in any one of Formulae (I) to (VIII), R³ is methyl. In certain embodiments, in any one of Formulae (I) to (VIII), R³ is C₁₋₆ heteroalkyl, optionally substituted with one or more substituents Q. In certain embodiments, in any one of Formulae (I) to (VIII), R³ is hydroxylmethyl, 2-hydroxylethyl, or 2-methoxyethyl.

In certain embodiments, in any one of Formulae (I) to (VIII), R⁴ is hydrogen. In certain embodiments, in any one of Formulae (I) to (VIII), R⁴ is deuterium. In certain embodiments, in any one of Formulae (I) to (VIII), R⁴ is cyano. In certain embodiments, in any one of Formulae (I) to (VIII), R⁴ is halo. In certain embodiments, in any one of Formulae (I) to (VIII), R⁴ is fluoro, chloro, or bromo. In certain embodiments, in any one of Formulae (I) to (VIII), R⁴ is fluoro. In certain embodiments, in any one of Formulae (I) to (VIII), R⁴ is chloro. In certain embodiments, in any one of Formulae (I) to (VIII), R⁴ is C₁₋₆ alkyl, optionally substituted with one or more substituents Q. In certain embodiments, in any one of Formulae (I) to (VIII), R⁴ is methyl, ethyl, or propyl. In certain embodiments, in any one of Formulae (I) to (VIII), R⁴ is methyl. In certain embodiments, in any one of Formulae (I) to (VIII), R⁴ is C₁₋₆ heteroalkyl, optionally substituted with one or more substituents Q. In certain embodiments, in any one of Formulae (I) to (VIII), R⁴ is hydroxylmethyl, 2-hydroxylethyl, or 2-methoxyethyl.

In certain embodiments, in any one of Formulae (I) to (VIII), R³ and R⁴ are each independently hydrogen, deuterium, cyano, fluoro, chloro, bromo, methyl, ethyl, n-propyl, isopropyl, hydroxylmethyl, 2-hydroxylethyl, or 2-methoxyethyl. In certain embodiments, in any one of Formulae (I) to (VIII), R³ and R⁴ are each independently hydrogen or deuterium. In certain embodiments, in any one of Formulae (I) to (VIII), R³ and R⁴ are each hydrogen.

In one embodiment, provided herein is a compound of Formula (IX):

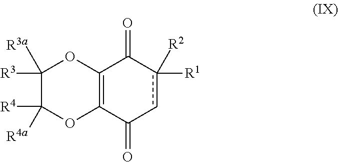

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein:
the symbol “ [symbol] ” represents a carbon-carbon single bond or double bond;
R¹ is (i) absent when the symbol “ [symbol] ” represents a carbon-carbon double bond or (ii) —SO₃H when the symbol “ [symbol] ” represents a carbon-carbon double bond; and
R², R³, R⁴, R^{3a}, and R^{4a} are each as defined herein.

In another embodiment, provided herein is a compound of Formula (X):

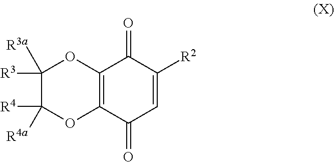

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R², R³, R⁴, R^{3a}, and R^{4a} are each as defined herein.

In yet another embodiment, provided herein is a compound of Formula (XI):

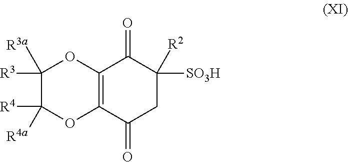

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R², R³, R⁴, R^{3a}, and R^{4a} are each as defined herein.

In still another embodiment, provided herein is a compound of Formula (XII):

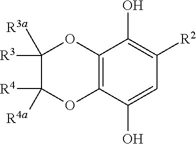

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R², R³, R⁴, R^{3a}, and R^{4a} are each as defined herein.

In certain embodiments, in any one of Formulae (A-II) and (IX) to (XII), R² is C₁₋₆ alkyl, optionally substituted with one or more substituents Q. In certain embodiments, in any one of Formulae (A-II) and (IX) to (XII), R² is methyl, ethyl, or propyl. In certain embodiments, in any one of Formulae (A-II) and (IX) to (XII), R² is methyl. In certain embodiments, in any one of Formulae (A-II) and (IX) to (XII), R² is C₁₋₆ heteroalkyl, optionally substituted with one or more substituents Q.

In one embodiment, provided herein is a compound of Formula (XIII):

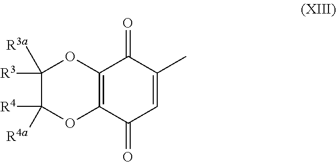

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R³, R⁴, R^{3a}, and R^{4a} are each as defined herein.

In another embodiment, provided herein is a compound of Formula (XIV):

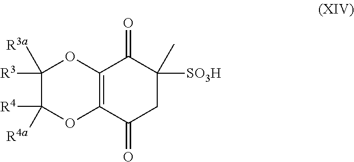

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R³, R⁴, R^{3a}, and R^{4a} are each as defined herein.

In yet another embodiment, provided herein is a compound of Formula (XV):

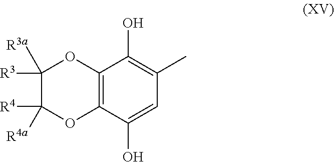

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R³, R⁴, R^{3a}, and R^{4a} are each as defined herein.

In certain embodiments, in any one of Formulae (A-II) and (IX) to (XV), R³ is hydrogen. In certain embodiments, in any one of Formulae (A-II) and (IX) to (XV), R³ is deuterium. In certain embodiments, in any one of Formulae (A-II) and (IX) to (XV), R³ is cyano. In certain embodiments, in any one of Formulae (A-II) and (IX) to (XV), R³ is halo. In certain embodiments, in any one of Formulae (A-II) and (IX) to (XV), R³ is fluoro, chloro, or bromo. In certain embodiments, in any one of Formulae (A-II) and (IX) to (XV), R³ is fluoro. In certain embodiments, in any one of Formulae (A-II) and (IX) to (XV), R³ is chloro. In certain embodiments, in any one of Formulae (A-II) and (IX) to (XV), R³ is C₁₋₆ alkyl, optionally substituted with one or more substituents Q. In certain embodiments, in any one of Formulae (A-II) and (IX) to (XV), R³ is methyl, ethyl, or propyl. In certain embodiments, in any one of Formulae (A-II) and (IX) to (XV), R³ is methyl. In certain embodiments, in any one of Formulae (A-II) and (IX) to (XV), R³ is C₁₋₆ heteroalkyl, optionally substituted with one or more substituents Q. In certain embodiments, in any one of Formulae (A-II) and (IX) to (XV), R³ is hydroxylmethyl, 2-hydroxylethyl, or 2-methoxyethyl.

In certain embodiments, in any one of Formulae (A-II) and (IX) to (XV), R⁴ is hydrogen. In certain embodiments, in any one of Formulae (A-II) and (IX) to (XV), R⁴ is deuterium. In certain embodiments, in any one of Formulae (A-II) and (IX) to (XV), R⁴ is cyano. In certain embodiments, in any one of Formulae (A-II) and (IX) to (XV), R⁴ is halo. In certain embodiments, in any one of Formulae (A-II) and (IX) to (XV), R⁴ is fluoro, chloro, or bromo. In certain embodiments, in any one of Formulae (A-II) and (IX) to (XV), R⁴ is fluoro. In certain embodiments, in any one of Formulae (A-II) and (IX) to (XV), R⁴ is chloro. In certain embodiments, in any one of Formulae (A-II) and (IX) to (XV), R⁴ is C₁₋₆ alkyl, optionally substituted with one or more substituents Q. In certain embodiments, in any one of Formulae (A-II) and (IX) to (XV), R⁴ is methyl, ethyl, or propyl. In certain embodiments, in any one of Formulae (A-II) and (IX) to (XV), R⁴ is methyl. In certain embodiments, in any one of Formulae (A-II) and (IX) to (XV), R⁴ is C₁₋₆ heteroalkyl, optionally substituted with one or more substituents Q. In certain embodiments, in any one of Formulae (A-II) and (IX) to (XV), R⁴ is hydroxylmethyl, 2-hydroxylethyl, or 2-methoxyethyl.

In certain embodiments, in any one of Formulae (A-II) and (IX) to (XV), R³ and R⁴ are each independently hydrogen, deuterium, cyano, fluoro, chloro, bromo, methyl, ethyl, n-propyl, isopropyl, hydroxylmethyl, 2-hydroxylethyl, or 2-methoxyethyl. In certain embodiments, in any one of Formulae (A-II) and (IX) to (XV), R³ and R⁴ are each independently hydrogen or deuterium. In certain embodiments, in any one of Formulae (A-II) and (IX) to (XV), R³ and R⁴ are each hydrogen.

In certain embodiments, in any one of Formulae (A-II) and (IX) to (XV), R^{3a} is hydrogen. In certain embodiments, in any one of Formulae (A-II) and (IX) to (XV), R^{3a} is deuterium. In certain embodiments, in any one of Formulae (A-II) and (IX) to (XV), R^{3a} is cyano. In certain embodiments, in any one of Formulae (A-II) and (IX) to (XV), R^{3a} is halo. In certain embodiments, in any one of Formulae (A-II) and (IX) to (XV), R^{3a} is fluoro, chloro, or bromo. In certain embodiments, in any one of Formulae (A-II) and (IX) to (XV), R^{3a} is fluoro. In certain embodiments, in any one of Formulae (A-II) and (IX) to (XV), R^{3a} is chloro. In certain embodiments, in any one of Formulae (A-II) and (IX) to (XV), R^{3a} is C₁₋₆ alkyl, optionally substituted with one or more substituents Q. In certain embodiments, in any one of Formulae (A-II) and (IX) to (XV), R^{3a} is methyl, ethyl, or propyl. In certain embodiments, in any one of Formulae (A-II) and (IX) to (XV), R^{3a} is methyl. In certain embodiments, in any one of Formulae (A-II) and (IX) to (XV), R^{3a} is C₁₋₆ heteroalkyl, optionally substituted with one or more substituents Q. In certain embodiments, in any one of Formulae (A-II) and (IX) to (XV), R^{3a} is hydroxylmethyl, 2-hydroxylethyl, or 2-methoxyethyl.

In certain embodiments, in any one of Formulae (A-II) and (IX) to (XV), R^{4a} is hydrogen. In certain embodiments, in any one of Formulae (A-II) and (IX) to (XV), R^{4a} is deuterium. In certain embodiments, in any one of Formulae (A-II) and (IX) to (XV), R^{4a} is cyano. In certain embodiments, in any one of Formulae (A-II) and (IX) to (XV), R^{4a} is halo. In certain embodiments, in any one of Formulae (A-II) and (IX) to (XV), R^{4a} is fluoro, chloro, or bromo. In certain embodiments, in any one of Formulae (A-II) and (IX) to (XV), R^{4a} is fluoro. In certain embodiments, in any one of Formulae (A-II) and (IX) to (XV), R^{4a} is chloro. In certain embodiments, in any one of Formulae (A-II) and (IX) to (XV), R^{4a} is C₁₋₆ alkyl, optionally substituted with one or more substituents Q. In certain embodiments, in any one of Formulae (A-II) and (IX) to (XV), R^{4a} is methyl, ethyl, or propyl. In certain embodiments, in any one of Formulae (A-II) and (IX) to (XV), R^{4a} is methyl. In certain embodiments, in any one of Formulae (A-II) and (IX) to (XV), R^{4a} is C₁₋₆ heteroalkyl, optionally substituted with one or more substituents Q. In certain embodiments, in any one of Formulae (A-II) and (IX) to (XV), R^{4a} is hydroxylmethyl, 2-hydroxylethyl, or 2-methoxyethyl.

In certain embodiments, in any one of Formulae (A-II) and (IX) to (XV), R^{3a} and R^{4a} are each independently hydrogen, deuterium, cyano, fluoro, chloro, bromo, methyl, ethyl, n-propyl, isopropyl, hydroxylmethyl, 2-hydroxylethyl, or 2-methoxyethyl. In certain embodiments, in any one of Formulae (A-II) and (IX) to (XV), R^{3a} and R^{4a} are each independently hydrogen or deuterium. In certain embodiments, in any one of Formulae (A-II) and (IX) to (XV), R^{3a} and R^{4a} are each hydrogen.

In certain embodiments, in any one of Formulae (A-II) and (IX) to (XV), R³, R⁴, R^{3a}, and R^{4a} are each independently hydrogen, deuterium, cyano, fluoro, chloro, bromo, methyl, ethyl, n-propyl, isopropyl, hydroxylmethyl, 2-hydroxylethyl, or 2-methoxyethyl. In certain embodiments, in any one of Formulae (A-II) and (IX) to (XV), R³, R⁴, R^{3a}, and R^{4a} are each independently hydrogen or deuterium. In certain embodiments, in any one of Formulae (A-II) and (IX) to (XV), R³, R⁴, R^{3a}, and R^{4a} are each hydrogen.

In one embodiment, provided herein is a compound of Formula (XVI):

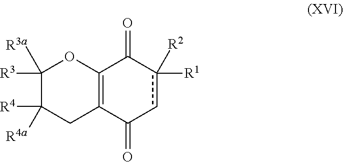

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R³, R⁴, R^{3a}, and R^{4a} are each as defined herein.

In another embodiment, provided herein is a compound of Formula (XVII):

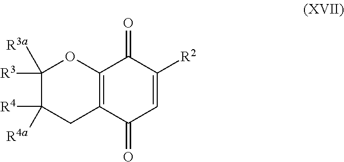

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R², R³, R⁴, R^{3a}, and R^{4a} are each as defined herein.

In yet another embodiment, provided herein is a compound of Formula (XVIII):

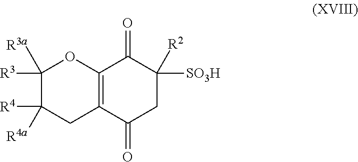

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R², R³, R⁴, R^{3a}, and R^{4a} are each as defined herein.

In still another embodiment, provided herein is a compound of Formula (XIX):

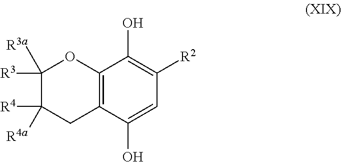

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R², R³, R⁴, R^{3a}, and R^{4a} are each as defined herein.

In certain embodiments, in any one of Formulae (A-I) and (XVI) to (XIX), R² is C₁₋₆ alkyl, optionally substituted with one or more substituents Q. In certain embodiments, in any one of Formulae (A-I) and (XVI) to (XIX), R² is methyl, ethyl, or propyl. In certain embodiments, in any one of Formulae (A-I) and (XVI) to (XIX), R² is methyl. In certain embodiments, in any one of Formulae (A-I) and (XVI) to (XIX), R² is C₁₋₆ heteroalkyl, optionally substituted with one or more substituents Q.

In one embodiment, provided herein is a compound of Formula (XX):

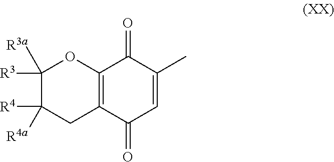

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R³, R⁴, R^{3a}, and R^{4a} are each as defined herein.

In another embodiment, provided herein is a compound of Formula (XXI):

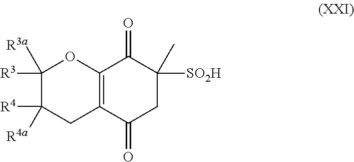

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R³, R⁴, R^{3a}, and R^{4a} are each as defined herein.

In yet another embodiment, provided herein is a compound of Formula (XXII):

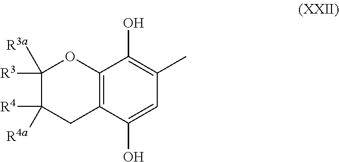

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R³, R⁴, R^{3a}, and R^{4a} are each as defined herein.

In one embodiment, provided herein is a compound of Formula (XXIII):

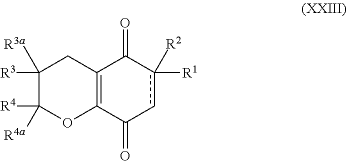

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R³, R⁴, R^{3a}, and R^{4a} are each as defined herein.

In another embodiment, provided herein is a compound of Formula (XXIV):

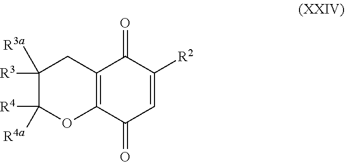

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R², R³, R⁴, R^{3a}, and R^{4a} are each as defined herein.

In yet another embodiment, provided herein is a compound of Formula (XXV):

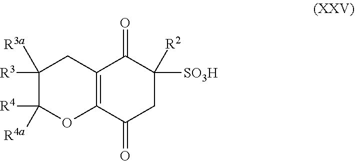

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R², R³, R⁴, R^{3a}, and R^{4a} are each as defined herein.

In still another embodiment, provided herein is a compound of Formula (XXVI):

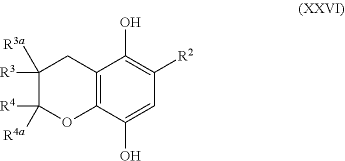

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R², R³, R⁴, R^{3a}, and R^{4a} are each as defined herein.

In certain embodiments, in any one of Formulae (XXIII) to (XXVI), R² is C₁₋₆ alkyl, optionally substituted with one or more substituents Q. In certain embodiments, in any one of Formulae (XXIII) to (XXVI), R² is methyl, ethyl, or propyl. In certain embodiments, in any one of Formulae (XXIII) to (XXVI), R² is methyl. In certain embodiments, in any one of Formulae (XXIII) to (XXVI), R² is C₁₋₆ heteroalkyl, optionally substituted with one or more substituents Q.

In one embodiment, provided herein is a compound of Formula (XXV):

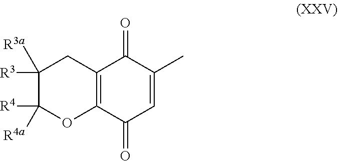

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R³, R⁴, R^{3a}, and R^{4a} are each as defined herein.

In another embodiment, provided herein is a compound of Formula (XXVI):

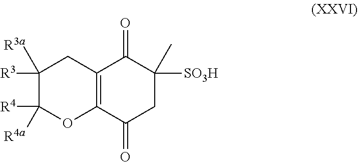

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R³, R⁴, R^{3a}, and R^{4a} are each as defined herein.

In yet another embodiment, provided herein is a compound of Formula (XXVII):

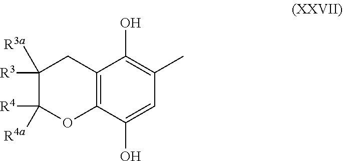

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R³, R⁴, R^{3a}, and R^{4a} are each as defined herein.

In certain embodiments, in any one of Formulae (A-I) and (IX) to (XXVII), R³ is hydrogen. In certain embodiments, in any one of Formulae (A-I) and (IX) to (XXVII), R³ is deuterium. In certain embodiments, in any one of Formulae (A-I) and (IX) to (XXVII), R³ is cyano. In certain embodiments, in any one of Formulae (A-I) and (IX) to (XXVII), R³ is halo. In certain embodiments, in any one of Formulae (A-I) and (IX) to (XXVII), R³ is fluoro, chloro, or bromo. In certain embodiments, in any one of Formulae (A-I) and (IX) to (XXVII), R³ is fluoro. In certain embodiments, in any one of Formulae (A-I) and (IX) to (XXVII), R³ is chloro. In certain embodiments, in any one of Formulae (A-I) and (IX) to (XXVII), R³ is C₁₋₆ alkyl, optionally substituted with one or more substituents Q. In certain embodiments, in any one of Formulae (A-I) and (IX) to (XXVII), R³ is methyl, ethyl, or propyl. In certain embodiments, in any one of Formulae (A-I) and (IX) to (XXVII), R³ is methyl. In certain embodiments, in any one of Formulae (A-I) and (IX) to (XXVII), R³ is C₁₋₆ heteroalkyl, optionally substituted with one or more substituents Q. In certain embodiments, in any one of Formulae (A-I) and (IX) to (XXVII), R³ is hydroxylmethyl, 2-hydroxylethyl, or 2-methoxyethyl.

In certain embodiments, in any one of Formulae (A-I) and (IX) to (XXVII), R⁴ is hydrogen. In certain embodiments, in any one of Formulae (A-I) and (IX) to (XXVII), R⁴ is deuterium. In certain embodiments, in any one of Formulae (A-I) and (IX) to (XXVII), R⁴ is cyano. In certain embodiments, in any one of Formulae (A-I) and (IX) to (XXVII), R⁴ is halo. In certain embodiments, in any one of Formulae (A-I) and (IX) to (XXVII), R⁴ is fluoro, chloro, or bromo. In certain embodiments, in any one of Formulae (A-I) and (IX) to (XXVII), R⁴ is fluoro. In certain embodiments, in any one of Formulae (A-I) and (IX) to (XXVII), R⁴ is chloro. In certain embodiments, in any one of Formulae (A-I) and (IX) to (XXVII), R⁴ is C₁₋₆ alkyl, optionally substituted with one or more substituents Q. In certain embodiments, in any one of Formulae (A-I) and (IX) to (XXVII), R⁴ is methyl, ethyl, or propyl. In certain embodiments, in any one of Formulae (A-I) and (IX) to (XXVII), R⁴ is methyl. In certain embodiments, in any one of Formulae (A-I) and (IX) to (XXVII), R⁴ is C₁₋₆ heteroalkyl, optionally substituted with one or more substituents Q. In certain embodiments, in any one of Formulae (A-I) and (IX) to (XXVII), R⁴ is hydroxylmethyl, 2-hydroxylethyl, or 2-methoxyethyl.

In certain embodiments, in any one of Formulae (A-I) and (IX) to (XXVII), R³ and R⁴ are each independently hydrogen, deuterium, cyano, fluoro, chloro, bromo, methyl, ethyl, n-propyl, isopropyl, hydroxylmethyl, 2-hydroxylethyl, or 2-methoxyethyl. In certain embodiments, in any one of Formulae (A-I) and (IX) to (XXVII), R³ and R⁴ are each independently hydrogen or deuterium. In certain embodiments, in any one of Formulae (A-I) and (IX) to (XXVII), R³ and R⁴ are each hydrogen.

In certain embodiments, in any one of Formulae (A-I) and (IX) to (XXVII), R^{3a} is hydrogen. In certain embodiments, in any one of Formulae (A-I) and (IX) to (XXVII), R^{3a} is deuterium. In certain embodiments, in any one of Formulae (A-I) and (IX) to (XXVII), R^{3a} is cyano. In certain embodiments, in any one of Formulae (A-I) and (IX) to (XXVII), R^{3a} is halo. In certain embodiments, in any one of Formulae (A-I) and (IX) to (XXVII), R^{3a} is fluoro, chloro, or bromo. In certain embodiments, in any one of Formulae (A-I) and (IX) to (XXVII), R^{3a} is fluoro. In certain embodiments, in any one of Formulae (A-I) and (IX) to (XXVII), R^{3a} is chloro. In certain embodiments, in any one of Formulae (A-I) and (IX) to (XXVII), R^{3a} is C₁₋₆ alkyl, optionally substituted with one or more substituents Q. In certain embodiments, in any one of Formulae (A-I) and (IX) to (XXVII), R^{3a} is methyl, ethyl, or propyl. In certain embodiments, in any one of Formulae (A-I) and (IX) to (XXVII), R^{3a} is methyl. In certain embodiments, in any one of Formulae (A-I) and (IX) to (XXVII), R^{3a} is C₁₋₆ heteroalkyl, optionally substituted with one or more substituents Q. In certain embodiments, in any one of Formulae (A-I) and (IX) to (XXVII), R^{3a} is hydroxylmethyl, 2-hydroxylethyl, or 2-methoxyethyl.

In certain embodiments, in any one of Formulae (A-I) and (IX) to (XXVII), R^{4a} is hydrogen. In certain embodiments, in any one of Formulae (A-I) and (IX) to (XXVII), R^{4a} is deuterium. In certain embodiments, in any one of Formulae (A-I) and (IX) to (XXVII), R^{4a} is cyano. In certain embodiments, in any one of Formulae (A-I) and (IX) to (XXVII), R^{4a} is halo. In certain embodiments, in any one of Formulae (A-I) and (IX) to (XXVII), R^{4a} is fluoro, chloro, or bromo. In certain embodiments, in any one of Formulae (A-I) and (IX) to (XXVII), R^{4a} is fluoro. In certain embodiments, in any one of Formulae (A-I) and (IX) to (XXVII), R^{4a} is chloro. In certain embodiments, in any one of Formulae (A-I) and (IX) to (XXVII), R^{4a} is C₁₋₆ alkyl, optionally substituted with one or more substituents Q. In certain embodiments, in any one of Formulae (A-I) and (IX) to (XXVII), R^{4a} is methyl, ethyl, or propyl. In certain embodiments, in any one of Formulae (A-I) and (IX) to (XXVII), R^{4a} is methyl. In certain embodiments, in any one of Formulae (A-I) and (IX) to (XXVII), R^{4a} is C₁₋₆ heteroalkyl, optionally substituted with one or more substituents Q. In certain embodiments, in any one of Formulae (A-I) and (IX) to (XXVII), R^{4a} is hydroxylmethyl, 2-hydroxylethyl, or 2-methoxyethyl.

In certain embodiments, in any one of Formulae (A-I) and (IX) to (XXVII), R^{3a} and R^{4a} are each independently hydrogen, deuterium, cyano, fluoro, chloro, bromo, methyl, ethyl, n-propyl, isopropyl, hydroxylmethyl, 2-hydroxylethyl, or 2-methoxyethyl. In certain embodiments, in any one of Formulae (A-I) and (IX) to (XXVII), R^{3a} and R^{4a} are each independently hydrogen or deuterium. In certain embodiments, in any one of Formulae (A-I) and (IX) to (XXVII), R^{3a} and R^{4a} are each hydrogen.

In certain embodiments, in any one of Formulae (A-I) and (IX) to (XXVII), R³, R⁴, R^{3a}, and R^{4a} are each independently hydrogen, deuterium, cyano, fluoro, chloro, bromo, methyl, ethyl, n-propyl, isopropyl, hydroxylmethyl, 2-hydroxylethyl, or 2-methoxyethyl. In certain embodiments, in any one of Formulae (A-I) and (IX) to (XXVII), R³, R⁴, R^{3a}, and R^{4a} are each independently hydrogen or deuterium. In certain embodiments, in any one of Formulae (A-I) and (IX) to (XXVII), R³, R⁴, R^{3a}, and R^{4a} are each hydrogen.

In one embodiment, provided herein is:
6-methylbenzo[b][1,4]dioxine-5,8-dione A1;
6-methyl-5,8-dioxo-5,6,7,8-tetrahydrobenzo[b][1,4]dioxine-6-sulfonic acid A2; or
6-methylbenzo[b][1,4]dioxine-5,8-diol A3;
   or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In another embodiment, provided herein is 6-methylbenzo[b][1,4]dioxine-5,8-dione A1; or a tautomer, a mixture of two or more tautomers, or an isotopic variant thereof; or a pharmaceutically acceptable salt, solvate, hydrate, or prodrug thereof.

In yet another embodiment, provided herein is 6-methyl-5,8-dioxo-5,6,7,8-tetrahydrobenzo[b][1,4]dioxine-6-sulfonic acid A2; or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In still another embodiment, provided herein is 6-methylbenzo[b][1,4]dioxine-5,8-diol A3; or a tautomer, a mixture of two or more tautomers, or an isotopic variant thereof, or a pharmaceutically acceptable salt, solvate, hydrate, or prodrug thereof.

In one embodiment, provided herein is:
6-methyl-2,3-dihydrobenzo[b][1,4]dioxine-5,8-dione B1;
6-methyl-5,8-dioxo-2,3,5,6,7,8-hexahydrobenzo[b][1,4]dioxine-6-sulfonic acid B2; or
6-methyl-2,3-dihydrobenzo[b][1,4]dioxine-5,8-diol B3;
   or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In another embodiment, provided herein is 6-methyl-2,3-dihydrobenzo[b][1,4]-dioxine-5,8-dione B1; or a tautomer, a mixture of two or more tautomers, or an isotopic variant thereof; or a pharmaceutically acceptable salt, solvate, hydrate, or prodrug thereof.

In yet another embodiment, provided herein is 6-methyl-5,8-dioxo-2,3,5,6,7,8-hexahydrobenzo[b][1,4]dioxine-6-sulfonic acid B2; or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In still another embodiment, provided herein is 6-methyl-2,3-dihydrobenzo[b][1,4]-dioxine-5,8-diol B3; or a tautomer, a mixture of two or more tautomers, or an isotopic variant thereof, or a pharmaceutically acceptable salt, solvate, hydrate, or prodrug thereof.

In one embodiment, provided herein is:
7-methyl-3,4-dihydro-2H-chromene-5,8-dione C1;
7-methyl-5,8-dioxo-3,4,5,6,7,8-hexahydro-2H-chromene-7-sulfonic acid C2; or
7-methylchromane-5,8-diol C3;
   or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In another embodiment, provided herein is 7-methyl-3,4-dihydro-2H-chromene-5,8-dione C1; or a tautomer, a mixture of two or more tautomers, or an isotopic variant thereof, or a pharmaceutically acceptable salt, solvate, hydrate, or prodrug thereof.

In yet another embodiment, provided herein is 7-methyl-5,8-dioxo-3,4,5,6,7,8-hexahydro-2H-chromene-7-sulfonic acid C2; or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In still another embodiment, provided herein is 7-methylchromane-5,8-diol C3; or a tautomer, a mixture of two or more tautomers, or an isotopic variant thereof; or a pharmaceutically acceptable salt, solvate, hydrate, or prodrug thereof.

In one embodiment, provided herein is:
6-methyl-3,4-dihydro-2H-chromene-5,8-dione D1;
6-methyl-5,8-dioxo-3,4,5,6,7,8-hexahydro-2H-chromene-6-sulfonic acid D2; or
   6-methylchromane-5,8-diol D3;
   or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In another embodiment, provided herein is 6-methyl-3,4-dihydro-2H-chromene-5,8-dione D1; or a tautomer, a mixture of two or more tautomers, or an isotopic variant thereof, or a pharmaceutically acceptable salt, solvate, hydrate, or prodrug thereof.

In yet another embodiment, provided herein is 6-methyl-5,8-dioxo-3,4,5,6,7,8-hexahydro-2H-chromene-6-sulfonic acid D2; or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In still another embodiment, provided herein is 6-methylchromane-5,8-diol D3; or a tautomer, a mixture of two or more tautomers, or an isotopic variant thereof; or a pharmaceutically acceptable salt, solvate, hydrate, or prodrug thereof.

In certain embodiments, a compound provided herein is deuterium-enriched. In certain embodiments, a compound provided herein is carbon-13 enriched. In certain embodiments, a compound provided herein is carbon-14 enriched. In certain embodiments, a compound provided herein contains one or more less prevalent isotopes for other elements, including, but not limited to, ¹⁵N for nitrogen; ¹⁷O or ¹⁸O for oxygen, and ³⁴S, ³⁵S, or ³⁶S for sulfur.

In certain embodiments, a compound provided herein has an isotopic enrichment factor of no less than about 5, no less than about 10, no less than about 20, no less than about 50, no less than about 100, no less than about 200, no less than about 500, no less than about 1,000, no less than about 2,000, no less than about 5,000, or no less than about 10,000. In any events, however, an isotopic enrichment factor for a specified isotope is no greater than the maximum isotopic enrichment factor for the specified isotope, which is the isotopic enrichment factor when a compound at a given position is 100% enriched with the specified isotope. Thus, the maximum isotopic enrichment factor is different for different isotopes. The maximum isotopic enrichment factor is 6,410 for deuterium and 90 for carbon-13.

In certain embodiments, a compound provided herein has a deuterium enrichment factor of no less than about 64 (about 1% deuterium enrichment), no less than about 130 (about 2% deuterium enrichment), no less than about 320 (about 5% deuterium enrichment), no less than about 640 (about 10% deuterium enrichment), no less than about 1,300 (about 20% deuterium enrichment), no less than about 3,200 (about 50% deuterium enrichment), no less than about 4,800 (about 75% deuterium enrichment), no less than about 5,130 (about 80% deuterium enrichment), no less than about 5,450 (about 85% deuterium enrichment), no less than about 5,770 (about 90% deuterium enrichment), no less than about 6,090 (about 95% deuterium enrichment), no less than about 6,220 (about 97% deuterium enrichment), no less than about 6,280 (about 98% deuterium enrichment), no less than about 6,350 (about 99% deuterium enrichment), or no less than about 6,380 (about 99.5% deuterium enrichment). The deuterium enrichment can be determined using conventional analytical methods known to one of ordinary skill in the art, including mass spectrometry and nuclear magnetic resonance spectroscopy. In certain embodiments, at least one of the atoms of a compound provided herein, as specified as deuterium-enriched, has deuterium enrichment of no less than about 50%, no less than about 70%, no less than about 80%, no less than about 90%, or no less than about 98%.

In certain embodiments, a compound provided herein is isolated or purified. In certain embodiments, a compound provided herein has a purity of at least about 90%, at least about 95%, at least about 98%, at least about 99%, or at least about 99.5% by weight.

The compounds provided herein are intended to encompass all possible stereoisomers unless a particular stereochemistry is specified. Where a compound provided herein contains an alkenyl group, the compound may exist as one or mixture of geometric cis/trans (or Z/E) isomers. Where structural isomers are interconvertible, the compound may exist as a single tautomer or a mixture of tautomers. This can take the form of proton tautomerism in the compound that contains, for example, an imino, keto, or oxime group; or so-called valence tautomerism in the compound that contains an aromatic moiety. It follows that a single compound may exhibit more than one type of isomerism.

A compound provided herein can be enantiomerically pure, such as a single enantiomer or a single diastereomer, or be stereoisomeric mixtures, such as a mixture of enantiomers, e.g., a racemic mixture of two enantiomers; or a mixture of two or more diastereomers. As such, one of ordinary skill in the art will recognize that administration of a compound in its (R) form is equivalent, for the compound that undergoes epimerization in vivo, to administration of the compound in its (S) form. Conventional techniques for the preparation/isolation of individual enantiomers include synthesis from a suitable optically pure precursor, asymmetric synthesis from achiral starting materials, or resolution of an enantiomeric mixture, for example, chiral chromatography, recrystallization, resolution, diastereomeric salt formation, or derivatization into diastereomeric adducts followed by separation.

When a compound provided herein contains an acidic or basic moiety, it can also be provided as a pharmaceutically acceptable salt. See, Berge et al., J. Pharm. Sci. 1977, 66, 1-19; Handbook of Pharmaceutical Salts: Properties, Selection, and Use, 2nd ed.; Stahl and Wermuth Eds.; John Wiley & Sons, 2011. In certain embodiments, a pharmaceutically acceptable salt of a compound provided herein is a solvate. In certain embodiments, a pharmaceutically acceptable salt of a compound provided herein is a hydrate.

Suitable acids for use in the preparation of pharmaceutically acceptable salts of a compound provided herein include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxoglutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts of a compound provided herein include, but are not limited to, inorganic bases, such as magnesium hydroxide, calcium hydroxide, potassium hydroxide, zinc hydroxide, and sodium hydroxide; and organic bases, such as primary, secondary, tertiary, and quaternary, aliphatic and aromatic amines, including, but not limited to, L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, triethanolamine, trimethylamine, triethylamine, N-methyl-D-glucamine, 2-amino-2-(hydroxymethyl)-1,3-propanediol, and tromethamine.

A compound provided herein may also be provided as a prodrug, which is a functional derivative of the compound and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis.

In one embodiment, provided herein is a pharmaceutically acceptable salt of 6-methyl-5,8-dioxo-5,6,7,8-tetrahydrobenzo[b][1,4]dioxine-6-sulfonic acid A2; or an enantiomer, a mixture of enantiomers, a diastereomer, a mixture of two or more diastereomers, a tautomer, a mixture of two or more tautomers, or an isotopic variant thereof, or a pharmaceutically acceptable solvate, hydrate, or prodrug thereof.

In another embodiment, provided herein is an alkali metal salt of 6-methyl-5,8-dioxo-5,6,7,8-tetrahydrobenzo[b][1,4]dioxine-6-sulfonic acid A2; or an enantiomer, a mixture of enantiomers, a diastereomer, a mixture of two or more diastereomers, a tautomer, a mixture of two or more tautomers, or an isotopic variant thereof; or a pharmaceutically acceptable solvate, hydrate, or prodrug thereof.

In yet another embodiment, provided herein is sodium or potassium 6-methyl-5,8-dioxo-5,6,7,8-tetrahydrobenzo[b][1,4]dioxine-6-sulfonate; or an enantiomer, a mixture of enantiomers, a diastereomer, a mixture of two or more diastereomers, a tautomer, a mixture of two or more tautomers, or an isotopic variant thereof; or a pharmaceutically acceptable solvate, hydrate, or prodrug thereof.

In yet another embodiment, provided herein is sodium 6-methyl-5,8-dioxo-5,6,7,8-tetrahydrobenzo[b][1,4]dioxine-6-sulfonate A4; or an enantiomer, a mixture of enantiomers, a diastereomer, a mixture of two or more diastereomers, a tautomer, a mixture of two or more tautomers, or an isotopic variant thereof; or a pharmaceutically acceptable solvate, hydrate, or prodrug thereof.

In yet another embodiment, provided herein is potassium 6-methyl-5,8-dioxo-5,6,7,8-tetrahydrobenzo[b][1,4]dioxine-6-sulfonate A5; or an enantiomer, a mixture of enantiomers, a diastereomer, a mixture of two or more diastereomers, a tautomer, a mixture of two or more tautomers, or an isotopic variant thereof; or a pharmaceutically acceptable solvate, hydrate, or prodrug thereof.

In yet another embodiment, provided herein is an alkaline earth metal salt of 6-methyl-5,8-dioxo-5,6,7,8-tetrahydrobenzo[b][1,4]dioxine-6-sulfonic acid A2; or an enantiomer, a mixture of enantiomers, a diastereomer, a mixture of two or more diastereomers, a tautomer, a mixture of two or more tautomers, or an isotopic variant thereof, or a pharmaceutically acceptable solvate, hydrate, or prodrug thereof.

In yet another embodiment, provided herein is calcium or magnesium 6-methyl-5,8-dioxo-5,6,7,8-tetrahydrobenzo[b][1,4]dioxine-6-sulfonate; or an enantiomer, a mixture of enantiomers, a diastereomer, a mixture of two or more diastereomers, a tautomer, a mixture of two or more tautomers, or an isotopic variant thereof; or a pharmaceutically acceptable solvate, hydrate, or prodrug thereof.

In yet another embodiment, provided herein is calcium 6-methyl-5,8-dioxo-5,6,7,8-tetrahydrobenzo[b][1,4]dioxine-6-sulfonate A6; or an enantiomer, a mixture of enantiomers, a diastereomer, a mixture of two or more diastereomers, a tautomer, a mixture of two or more tautomers, or an isotopic variant thereof; or a pharmaceutically acceptable solvate, hydrate, or prodrug thereof.

In still another embodiment, provided herein is magnesium 6-methyl-5,8-dioxo-5,6,7,8-tetrahydrobenzo[b][1,4]dioxine-6-sulfonate A7; or an enantiomer, a mixture of enantiomers, a diastereomer, a mixture of two or more diastereomers, a tautomer, a mixture of two or more tautomers, or an isotopic variant thereof; or a pharmaceutically acceptable solvate, hydrate, or prodrug thereof.

In one embodiment, provided herein is a pharmaceutically acceptable salt of 6-methyl-5,8-dioxo-2,3,5,6,7,8-hexahydrobenzo[b][1,4]dioxine-6-sulfonic acid B2; or an enantiomer, a mixture of enantiomers, a diastereomer, a mixture of two or more diastereomers, a tautomer, a mixture of two or more tautomers, or an isotopic variant thereof; or a pharmaceutically acceptable solvate, hydrate, or prodrug thereof.

In another embodiment, provided herein is an alkali metal salt of 6-methyl-5,8-dioxo-2,3,5,6,7,8-hexahydrobenzo[b][1,4]dioxine-6-sulfonic acid B2; or an enantiomer, a mixture of enantiomers, a diastereomer, a mixture of two or more diastereomers, a tautomer, a mixture of two or more tautomers, or an isotopic variant thereof, or a pharmaceutically acceptable solvate, hydrate, or prodrug thereof.

In yet another embodiment, provided herein is sodium or potassium 6-methyl-5,8-dioxo-2,3,5,6,7,8-hexahydrobenzo[b][1,4]dioxine-6-sulfonate; or an enantiomer, a mixture of enantiomers, a diastereomer, a mixture of two or more diastereomers, a tautomer, a mixture of two or more tautomers, or an isotopic variant thereof; or a pharmaceutically acceptable solvate, hydrate, or prodrug thereof.

In yet another embodiment, provided herein is sodium 6-methyl-5,8-dioxo-2,3,5,6,7,8-hexahydrobenzo[b][1,4]dioxine-6-sulfonate B4; or an enantiomer, a mixture of enantiomers, a diastereomer, a mixture of two or more diastereomers, a tautomer, a mixture of two or more tautomers, or an isotopic variant thereof; or a pharmaceutically acceptable solvate, hydrate, or prodrug thereof.

In yet another embodiment, provided herein is potassium 6-methyl-5,8-dioxo-2,3,5,6,7,8-hexahydrobenzo[b][1,4]dioxine-6-sulfonate B5; or an enantiomer, a mixture of enantiomers, a diastereomer, a mixture of two or more diastereomers, a tautomer, a mixture of two or more tautomers, or an isotopic variant thereof; or a pharmaceutically acceptable solvate, hydrate, or prodrug thereof.

In yet another embodiment, provided herein is an alkaline earth metal salt of 6-methyl-5,8-dioxo-2,3,5,6,7,8-hexahydrobenzo[b][1,4]dioxine-6-sulfonic acid B2; or an enantiomer, a mixture of enantiomers, a diastereomer, a mixture of two or more diastereomers, a tautomer, a mixture of two or more tautomers, or an isotopic variant thereof; or a pharmaceutically acceptable solvate, hydrate, or prodrug thereof.

In yet another embodiment, provided herein is calcium or magnesium 6-methyl-5,8-dioxo-2,3,5,6,7,8-hexahydrobenzo[b][1,4]dioxine-6-sulfonate; or an enantiomer, a mixture of enantiomers, a diastereomer, a mixture of two or more diastereomers, a tautomer, a mixture of two or more tautomers, or an isotopic variant thereof; or a pharmaceutically acceptable solvate, hydrate, or prodrug thereof.

In yet another embodiment, provided herein is calcium 6-methyl-5,8-dioxo-2,3,5,6,7,8-hexahydrobenzo[b][1,4]dioxine-6-sulfonate B6; or an enantiomer, a mixture of enantiomers, a diastereomer, a mixture of two or more diastereomers, a tautomer, a mixture of two or more tautomers, or an isotopic variant thereof; or a pharmaceutically acceptable solvate, hydrate, or prodrug thereof.

In still another embodiment, provided herein is magnesium 6-methyl-5,8-dioxo-2,3,5,6,7,8-hexahydrobenzo[b][1,4]dioxine-6-sulfonate B7; or an enantiomer, a mixture of enantiomers, a diastereomer, a mixture of two or more diastereomers, a tautomer, a mixture of two or more tautomers, or an isotopic variant thereof; or a pharmaceutically acceptable solvate, hydrate, or prodrug thereof.

In one embodiment, provided herein is a pharmaceutically acceptable salt of 7-methyl-5,8-dioxo-3,4,5,6,7,8-hexahydro-2H-chromene-7-sulfonic acid C2; or an enantiomer, a mixture of enantiomers, a diastereomer, a mixture of two or more diastereomers, a tautomer, a mixture of two or more tautomers, or an isotopic variant thereof; or a pharmaceutically acceptable solvate, hydrate, or prodrug thereof.

In another embodiment, provided herein is an alkali metal salt of 7-methyl-5,8-dioxo-3,4,5,6,7,8-hexahydro-2H-chromene-7-sulfonic acid C2; or an enantiomer, a mixture of enantiomers, a diastereomer, a mixture of two or more diastereomers, a tautomer, a mixture of two or more tautomers, or an isotopic variant thereof; or a pharmaceutically acceptable solvate, hydrate, or prodrug thereof.

In yet another embodiment, provided herein is sodium or potassium 7-methyl-5,8-dioxo-3,4,5,6,7,8-hexahydro-2H-chromene-7-sulfonate; or an enantiomer, a mixture of enantiomers, a diastereomer, a mixture of two or more diastereomers, a tautomer, a mixture of two or more tautomers, or an isotopic variant thereof; or a pharmaceutically acceptable solvate, hydrate, or prodrug thereof.

In yet another embodiment, provided herein is sodium 7-methyl-5,8-dioxo-3,4,5,6,7,8-hexahydro-2H-chromene-7-sulfonate C4; or an enantiomer, a mixture of enantiomers, a diastereomer, a mixture of two or more diastereomers, a tautomer, a mixture of two or more tautomers, or an isotopic variant thereof; or a pharmaceutically acceptable solvate, hydrate, or prodrug thereof.

In yet another embodiment, provided herein is potassium 7-methyl-5,8-dioxo-3,4,5,6,7,8-hexahydro-2H-chromene-7-sulfonate C5; or an enantiomer, a mixture of enantiomers, a diastereomer, a mixture of two or more diastereomers, a tautomer, a mixture of two or more tautomers, or an isotopic variant thereof; or a pharmaceutically acceptable solvate, hydrate, or prodrug thereof.

In yet another embodiment, provided herein is an alkaline earth metal salt of 7-methyl-5,8-dioxo-3,4,5,6,7,8-hexahydro-2H-chromene-7-sulfonic acid C2; or an enantiomer, a mixture of enantiomers, a diastereomer, a mixture of two or more diastereomers, a tautomer, a mixture of two or more tautomers, or an isotopic variant thereof, or a pharmaceutically acceptable solvate, hydrate, or prodrug thereof.

In yet another embodiment, provided herein is calcium or magnesium 7-methyl-5,8-dioxo-3,4,5,6,7,8-hexahydro-2H-chromene-7-sulfonate; or an enantiomer, a mixture of enantiomers, a diastereomer, a mixture of two or more diastereomers, a tautomer, a mixture of two or more tautomers, or an isotopic variant thereof; or a pharmaceutically acceptable solvate, hydrate, or prodrug thereof.

In yet another embodiment, provided herein is calcium 7-methyl-5,8-dioxo-3,4,5,6,7,8-hexahydro-2H-chromene-7-sulfonate C6; or an enantiomer, a mixture of enantiomers, a diastereomer, a mixture of two or more diastereomers, a tautomer, a mixture of two or more tautomers, or an isotopic variant thereof; or a pharmaceutically acceptable solvate, hydrate, or prodrug thereof.

In still another embodiment, provided herein is magnesium 7-methyl-5,8-dioxo-3,4,5,6,7,8-hexahydro-2H-chromene-7-sulfonate C7; or an enantiomer, a mixture of enantiomers, a diastereomer, a mixture of two or more diastereomers, a tautomer, a mixture of two or more tautomers, or an isotopic variant thereof; or a pharmaceutically acceptable solvate, hydrate, or prodrug thereof.

In one embodiment, provided herein is a pharmaceutically acceptable salt of 6-methyl-5,8-dioxo-3,4,5,6,7,8-hexahydro-2H-chromene-6-sulfonic acid D2; or an enantiomer, a mixture of enantiomers, a diastereomer, a mixture of two or more diastereomers, a tautomer, a mixture of two or more tautomers, or an isotopic variant thereof, or a pharmaceutically acceptable solvate, hydrate, or prodrug thereof.

In another embodiment, provided herein is an alkali metal salt of 6-methyl-5,8-dioxo-3,4,5,6,7,8-hexahydro-2H-chromene-6-sulfonic acid D2; or an enantiomer, a mixture of enantiomers, a diastereomer, a mixture of two or more diastereomers, a tautomer, a mixture of two or more tautomers, or an isotopic variant thereof; or a pharmaceutically acceptable solvate, hydrate, or prodrug thereof.

In yet another embodiment, provided herein is sodium or potassium 6-methyl-5,8-dioxo-3,4,5,6,7,8-hexahydro-2H-chromene-6-sulfonate; or an enantiomer, a mixture of enantiomers, a diastereomer, a mixture of two or more diastereomers, a tautomer, a mixture of two or more tautomers, or an isotopic variant thereof; or a pharmaceutically acceptable solvate, hydrate, or prodrug thereof.

In yet another embodiment, provided herein is sodium 6-methyl-5,8-dioxo-3,4,5,6,7,8-hexahydro-2H-chromene-6-sulfonate D4; or an enantiomer, a mixture of enantiomers, a diastereomer, a mixture of two or more diastereomers, a tautomer, a mixture of two or more tautomers, or an isotopic variant thereof; or a pharmaceutically acceptable solvate, hydrate, or prodrug thereof.

In yet another embodiment, provided herein is potassium 6-methyl-5,8-dioxo-3,4,5,6,7,8-hexahydro-2H-chromene-6-sulfonate D5; or an enantiomer, a mixture of enantiomers, a diastereomer, a mixture of two or more diastereomers, a tautomer, a mixture of two or more tautomers, or an isotopic variant thereof; or a pharmaceutically acceptable solvate, hydrate, or prodrug thereof.

In yet another embodiment, provided herein is an alkaline earth metal salt of 6-methyl-5,8-dioxo-3,4,5,6,7,8-hexahydro-2H-chromene-6-sulfonic acid D2; or an enantiomer, a mixture of enantiomers, a diastereomer, a mixture of two or more diastereomers, a tautomer, a mixture of two or more tautomers, or an isotopic variant thereof, or a pharmaceutically acceptable solvate, hydrate, or prodrug thereof.

In yet another embodiment, provided herein is calcium or magnesium 6-methyl-5,8-dioxo-3,4,5,6,7,8-hexahydro-2H-chromene-6-sulfonate; or an enantiomer, a mixture of enantiomers, a diastereomer, a mixture of two or more diastereomers, a tautomer, a mixture of two or more tautomers, or an isotopic variant thereof; or a pharmaceutically acceptable solvate, hydrate, or prodrug thereof.

In yet another embodiment, provided herein is calcium 6-methyl-5,8-dioxo-3,4,5,6,7,8-hexahydro-2H-chromene-6-sulfonate D6; or an enantiomer, a mixture of enantiomers, a diastereomer, a mixture of two or more diastereomers, a tautomer, a mixture of two or more tautomers, or an isotopic variant thereof; or a pharmaceutically acceptable solvate, hydrate, or prodrug thereof.

In still another embodiment, provided herein is magnesium 6-methyl-5,8-dioxo-3,4,5,6,7,8-hexahydro-2H-chromene-6-sulfonate D7; or an enantiomer, a mixture of enantiomers, a diastereomer, a mixture of two or more diastereomers, a tautomer, a mixture of two or more tautomers, or an isotopic variant thereof; or a pharmaceutically acceptable solvate, hydrate, or prodrug thereof.

### Pharmaceutical Compositions

In one embodiment, provided herein is a pharmaceutical composition, comprising a compound provided herein, e.g., a compound of Formula (A-I), (A-II), or (I), or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof; and a pharmaceutically acceptable excipient.

The pharmaceutical composition provided herein can be formulated in various dosage forms, including, but not limited to, dosage forms for oral, parenteral, and topical administration. The pharmaceutical composition can also be formulated as modified release dosage forms, including delayed-, extended-, prolonged-, sustained-, pulsatile-, controlled-, accelerated-, fast-, targeted-, programmed-release, and gastric retention dosage forms. These dosage forms can be prepared according to conventional methods and techniques known to those skilled in the art. See, e.g., Remington: The Science and Practice of Pharmacy, supra; Modified-Release Drug Delivery Technology, 2nd ed.; Rathbone et al., Eds.; Drugs and the Pharmaceutical Sciences 184; CRC Press: Boca Raton, FL, 2008.

In one embodiment, the pharmaceutical composition provided herein is formulated in a dosage form for oral administration. In another embodiment, the pharmaceutical composition provided herein is formulated in a dosage form for parenteral administration. In yet another embodiment, the pharmaceutical composition provided herein is formulated in a dosage form for intravenous administration. In yet another embodiment, the pharmaceutical composition provided herein is formulated in a dosage form for intramuscular administration. In yet another embodiment, the pharmaceutical composition provided herein is formulated in a dosage form for subcutaneous administration. In still another embodiment, the pharmaceutical composition provided herein is formulated in a dosage form for topical administration.

The pharmaceutical composition provided herein can be provided in a unit-dosage form or multiple-dosage form. A unit-dosage form, as used herein, refers to physically discrete a unit suitable for administration to a subject, and packaged individually as is known in the art. Each unit-dose contains a predetermined quantity of an active ingredient(s) (e.g., a compound provided herein) sufficient to produce the desired therapeutic effect, in association with the required pharmaceutical excipient(s). Examples of a unit-dosage form include, but are not limited to, an ampoule, syringe, and individually packaged tablet and capsule. A unit-dosage form may be administered in fractions or multiples thereof. A multiple-dosage form is a plurality of identical unit-dosage forms packaged in a single container to be administered in a segregated unit-dosage form. Examples of a multiple-dosage form include, are not limited to, a vial, bottle of tablets or capsules, or bottle of pints or gallons.

The pharmaceutical composition provided herein can be administered at once or multiple times at intervals of time. It is understood that the precise dosage and duration of treatment may vary with the age, weight, and condition of the subject being treated, and may be determined empirically using known testing protocols or by extrapolation from in vivo or in vitro test or diagnostic data. It is further understood that for any particular individual, specific dosage regimens should be adjusted over time according to the subject's need and the professional judgment of the person administering or supervising the administration of the pharmaceutical composition.

### A. Oral Administration

The pharmaceutical composition provided herein for oral administration can be provided in solid, semisolid, or liquid dosage forms for oral administration. As used herein, oral administration also includes buccal, lingual, and sublingual administration. Suitable oral dosage forms include, but are not limited to, tablets, fastmelts, chewable tablets, capsules, pills, strips, troches, lozenges, pastilles, cachets, pellets, medicated chewing gum, bulk powders, effervescent or non-effervescent powders or granules, oral mists, solutions, emulsions, suspensions, wafers, sprinkles, elixirs, and syrups. In addition to the active ingredient(s), the pharmaceutical composition can contain one or more pharmaceutically acceptable carriers or excipients, including, but not limited to, binders, fillers, diluents, disintegrants, wetting agents, lubricants, glidants, coloring agents, dye-migration inhibitors, sweetening agents, flavoring agents, emulsifying agents, suspending and dispersing agents, preservatives, solvents, non-aqueous liquids, organic acids, and sources of carbon dioxide.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500®); gelatin; sugars, such as sucrose, glucose, dextrose, molasses, and lactose; natural and synthetic gums, such as acacia, alginic acid, alginates, extract of Irish moss, Panwar gum, Ghatti gum, mucilage of isabgol husks, carboxymethylcellulose, methylcellulose, polyvinylpyrrolidone (PVP), VEEGUM®, larch arabinogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); and microcrystalline celluloses, such as AVICEL® PH-101, AVICEL® PH-103, AVICEL® PH-105, and AVICEL® RC-581. Suitable fillers include, but are not limited to, talc, calcium carbonate, microcrystalline cellulose, powdered cellulose, dextrates, kaolin, mannitol, silicic acid, sorbitol, starch, and pre-gelatinized starch. The amount of a binder or filler in the pharmaceutical composition provided herein varies upon the type of formulation, and is readily discernible to those of ordinary skill in the art. The binder or filler may be present from about 50 to about 99% by weight in the pharmaceutical composition provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets. The amount of a diluent in the pharmaceutical composition provided herein varies upon the type of formulation, and is readily discernible to those of ordinary skill in the art.

Suitable disintegrants include, but are not limited to, agar; bentonite; celluloses, such as methylcellulose and carboxymethylcellulose; wood products; natural sponge; cation-exchange resins; alginic acid; gums, such as guar gum and VEEGUM® HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; and algins. The amount of a disintegrant in the pharmaceutical composition provided herein varies upon the type of formulation, and is readily discernible to those of ordinary skill in the art. The pharmaceutical composition provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, such as peanut oil, cottonseed oil, sunflower oil, sesame oil, olive oil, corn oil, and soybean oil; zinc stearate; ethyl oleate; ethyl laureate; agar; starch; lycopodium; and silica or silica gels, such as AEROSII® ® 200 and CAB-O-SIL®. The amount of a lubricant in the pharmaceutical composition provided herein varies upon the type of formulation, and is readily discernible to those of ordinary skill in the art. The pharmaceutical compositions provided herein may contain about 0.1 to about 5% by weight of a lubricant.

Suitable glidants include, but are not limited to, colloidal silicon dioxide, CAB—O-SIL®, and asbestos-free talc. Suitable coloring agents include, but are not limited to, any of the approved, certified, water soluble FD&C dyes, and water insoluble FD&C dyes suspended on alumina hydrate, and color lakes. A color lake is a combination by adsorption of a water-soluble dye to a hydrous oxide of a heavy metal, resulting in an insoluble form of the dye. Suitable flavoring agents include, but are not limited to, natural flavors extracted from plants, such as fruits, and synthetic blends of compounds which produce a pleasant taste sensation, such as peppermint and methyl salicylate. Suitable sweetening agents include, but are not limited to, sucrose, lactose, mannitol, syrups, glycerin, and artificial sweeteners, such as saccharin and aspartame. Suitable emulsifying agents include, but are not limited to, gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suitable suspending and dispersing agents include, but are not limited to, sodium carboxymethylcellulose, pectin, tragacanth, VEEGUM®, acacia, sodium carboxymethylcellulose, hydroxypropyl methylcellulose, and polyvinylpyrrolidone. Suitable preservatives include, but are not limited to, glycerin, methyl and propylparaben, benzoic add, and sodium benzoate and alcohol. Suitable wetting agents include, but are not limited to, propylene glycol monostearate, sorbitan monooleate, diethylene glycol monolaurate, and polyoxyethylene lauryl ether. Suitable solvents include, but are not limited to, glycerin, sorbitol, ethyl alcohol, and syrup. Suitable non-aqueous liquids utilized in emulsions include, but are not limited to, mineral oil and cottonseed oil. Suitable organic acids include, but are not limited to, citric and tartaric acid. Suitable sources of carbon dioxide include, but are not limited to, sodium bicarbonate and sodium carbonate.

It should be understood that many carriers and excipients may serve several functions, even within the same formulation.

The pharmaceutical composition provided herein for oral administration can be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredient(s) from the acidic environment of the stomach. Enteric-coatings include, but are not limited to, fatty acids, fats, phenyl salicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms can be prepared from an active ingredient(s) in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical composition provided herein for oral administration can be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient(s). The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient(s).

The pharmaceutical composition provided herein for oral administration can be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquid or solvent, emulsifying agent, and preservative. Suspensions may include a pharmaceutically acceptable suspending agent and preservative. Aqueous alcoholic solutions may include a pharmaceutically acceptable acetal, such as a di(lower alkyl) acetal of a lower alkyl aldehyde, e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing an active ingredient(s), and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These dosage forms can further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

The pharmaceutical composition provided herein for oral administration can be also provided in the forms of liposomes, micelles, microspheres, or nanosystems. Micellar dosage forms can be prepared as described in U.S. Pat. No. 6,350,458.

The pharmaceutical composition provided herein for oral administration can be provided as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all of the dosage forms described herein.

The pharmaceutical composition provided herein for oral administration can be formulated as immediate or modified release dosage forms, including delayed-, sustained, pulsed-, controlled, targeted-, and programmed-release forms.

### B. Parenteral Administration

The pharmaceutical composition provided herein can be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, intravesical, and subcutaneous administration.

The pharmaceutical composition provided herein for parenteral administration can be formulated in any dosage forms that are suitable for parenteral administration, including, but not limited to, solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science. See, e.g., Remington: The Science and Practice of Pharmacy, supra.

The pharmaceutical composition provided herein for parenteral administration can include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringer's injection, isotonic dextrose injection, sterile water injection, dextrose and lactated Ringer's injection. Suitable non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Suitable water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, N,N-dimethylacetamide, and dimethyl sulfoxide.

Suitable antimicrobial agents or preservatives include, but are not limited to, phenols, cresols, mercurials, benzyl alcohol, chlorobutanol, methyl and propyl p-hydroxy-benzoates, thimerosal, benzalkonium chloride (e.g., benzethonium chloride), methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants include those described herein, such as bisulfite and sodium metabisulfite. Suitable local anesthetics include, but are not limited to, procaine hydrochloride. Suitable suspending and dispersing agents include those described herein, such as sodium carboxymethylcellulose, hydroxypropyl methylcellulose, and polyvinylpyrrolidone. Suitable emulsifying agents include those described herein, such as polyoxyethylene sorbitan monolaurate, polyoxyethylene sorbitan monooleate 80, and triethanolamine oleate. Suitable sequestering or chelating agents include, but are not limited to, EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®).

When the pharmaceutical composition provided herein is formulated for multiple dosage administration, multiple dosage parenteral formulations must contain an antimicrobial agent at bacteriostatic or fungistatic concentrations. All parenteral formulations must be sterile, as known and practiced in the art.

In one embodiment, the pharmaceutical composition for parenteral administration is provided as a ready-to-use sterile solution. In another embodiment, the pharmaceutical composition is provided as a sterile dry soluble product, including a lyophilized powder and hypodermic tablet, to be reconstituted with a vehicle prior to use. In yet another embodiment, the pharmaceutical composition is provided as a ready-to-use sterile suspension. In yet another embodiment, the pharmaceutical composition is provided as a sterile dry insoluble product to be reconstituted with a vehicle prior to use. In still another embodiment, the pharmaceutical composition is provided as a ready-to-use sterile emulsion.

The pharmaceutical composition provided herein for parenteral administration can be formulated as immediate or modified release dosage forms, including delayed-, sustained, pulsed-, controlled, targeted-, and programmed-release forms.

The pharmaceutical composition provided herein for parenteral administration can be formulated as a suspension, solid, semi-solid, or thixotropic liquid, for administration as an implanted depot. In one embodiment, the pharmaceutical composition provided herein are dispersed in a solid inner matrix, which is surrounded by an outer polymeric membrane that is insoluble in body fluids but allows the active ingredient(s) in the pharmaceutical composition to diffuse through.

Suitable inner matrixes include, but are not limited to, polymethylmethacrylate, polybutylmethacrylate, plasticized or unplasticized polyvinylchloride, plasticized nylon, plasticized polyethylene terephthalate, natural rubber, polyisoprene, polyisobutylene, polybutadiene, polyethylene, ethylene-vinyl acetate copolymers, silicone rubbers, polydimethylsiloxanes, silicone carbonate copolymers, hydrophilic polymers (such as hydrogels of esters of acrylic and methacrylic acid), collagen, cross-linked polyvinyl alcohol, and cross-linked partially hydrolyzed polyvinyl acetate.

Suitable outer polymeric membranes include, but are not limited to, polyethylene, polypropylene, ethylene/propylene copolymers, ethylene/ethyl acrylate copolymers, ethylene/vinyl acetate copolymers, silicone rubbers, polydimethylsiloxanes, neoprene rubber, chlorinated polyethylene, polyvinylchloride, vinyl chloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

### C. Topical Administration

The pharmaceutical composition provided herein can be administered topically to the skin, orifices, or mucosa. The topical administration, as used herein, includes (intra)dermal, conjunctival, intracorneal, intraocular, ophthalmic, auricular, transdermal, nasal, vaginal, urethral, respiratory, and rectal administration.

The pharmaceutical composition provided herein can be formulated in any dosage forms that are suitable for topical administration for local or systemic effect, including, but not limited to, emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, and dermal patches. The topical formulations of the pharmaceutical composition provided herein can also comprise liposomes, micelles, microspheres, and nanosystems.

Pharmaceutically acceptable carriers and excipients suitable for use in the topical formulations include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryoprotectants, lyoprotectants, thickening agents, and inert gases.

The pharmaceutical composition can also be administered topically by electroporation, iontophoresis, phonophoresis, sonophoresis, or microneedle or needle-free injection, such as POWDERJECT™ and BIOJECT™.

The pharmaceutical composition provided herein can be provided in the forms of ointments, creams, and gels. Suitable ointment vehicles include oleaginous or hydrocarbon vehicles, including lard, benzoinated lard, olive oil, cottonseed oil, and other oils, white petrolatum; emulsifiable or absorption vehicles, such as hydrophilic petrolatum, hydroxystearin sulfate, and anhydrous lanolin; water-removable vehicles, such as hydrophilic ointment; water-soluble ointment vehicles, including polyethylene glycols of varying molecular weight; emulsion vehicles, either water-in-oil (W/O) emulsions or oil-in-water (O/W) emulsions, including cetyl alcohol, glyceryl monostearate, lanolin, and stearic acid. See, e.g., Remington: The Science and Practice ofPharmacy, supra. These vehicles are emollient but generally require addition of antioxidants and preservatives.

Suitable cream base can be oil-in-water or water-in-oil. Suitable cream vehicles may be water-washable, and contain an oil phase, an emulsifier, and an aqueous phase. The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation may be a nonionic, anionic, cationic, or amphoteric surfactant.

Gels are semisolid, suspension-type systems. Single-phase gels contain organic macromolecules distributed substantially uniformly throughout the liquid carrier. Suitable gelling agents include, but are not limited to, crosslinked acrylic acid polymers, such as carbomers, carboxypolyalkylenes, and CARBOPOL®; hydrophilic polymers, such as polyethylene oxides, polyoxyethylene-polyoxypropylene copolymers, and polyvinylalcohol; cellulosic polymers, such as hydroxypropyl cellulose, hydroxyethyl cellulose, hydroxypropyl methylcellulose, hydroxypropyl methylcellulose phthalate, and methylcellulose; gums, such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

The pharmaceutical composition provided herein can be administered rectally, urethrally, vaginally, or perivaginally in the forms of suppositories, pessaries, bougies, poultices or cataplasm, pastes, powders, dressings, creams, plasters, contraceptives, ointments, solutions, emulsions, suspensions, tampons, gels, foams, sprays, or enemas. These dosage forms can be manufactured using conventional processes as described in Remington: The Science andPractice of Pharmacy, supra.

Rectal, urethral, and vaginal suppositories are solid bodies for insertion into body orifices, which are solid at ordinary temperatures but melt or soften at body temperature to release the active ingredient(s) inside the orifices. Pharmaceutically acceptable carriers utilized in rectal and vaginal suppositories include bases or vehicles, such as stiffening agents, which produce a melting point in the proximity of body temperature, when formulated with an active ingredient(s); and antioxidants as described herein, including bisulfite and sodium metabisulfite. Suitable vehicles include, but are not limited to, cocoa butter (theobroma oil), glycerin-gelatin, carbowax (polyoxyethylene glycol), spermaceti, paraffin, white and yellow wax, and appropriate mixtures of mono-, di- and triglycerides of fatty acids, and hydrogels, such as polyvinyl alcohol, hydroxyethyl methacrylate, and polyacrylic acid. Combinations of the various vehicles can also be used. Rectal and vaginal suppositories may be prepared by compressing or molding. The typical weight of a rectal and vaginal suppository is about 2 to about 3 g.

The pharmaceutical composition provided herein can be administered ophthalmically in the forms of solutions, suspensions, ointments, emulsions, gel-forming solutions, powders for solutions, gels, ocular inserts, and implants.

The pharmaceutical composition provided herein can be administered intranasally or by inhalation to the respiratory tract. The pharmaceutical composition can be provided in the form of an aerosol or solution for delivery using a pressurized container, pump, spray, atomizer, such as an atomizer using electrohydrodynamics to produce a fine mist, or nebulizer, alone or in combination with a suitable propellant, such as 1,1,1,2-tetrafluoroethane or 1,1,1,2,3,3,3-heptafluoropropane. The pharmaceutical composition can also be provided as a dry powder for insufflation, alone or in combination with an inert carrier such as lactose or phospholipids; and nasal drops. For intranasal use, the powder can comprise a bioadhesive agent, including chitosan or cyclodextrin.

Solutions or suspensions for use in a pressurized container, pump, spray, atomizer, or nebulizer can be formulated to contain ethanol, aqueous ethanol, or a suitable alternative agent for dispersing, solubilizing, or extending release of an active ingredient(s); a propellant as solvent; and/or a surfactant, such as sorbitan trioleate, oleic acid, or an oligolactic acid.

The pharmaceutical composition provided herein can be micronized to a size suitable for delivery by inhalation, such as about 50 micrometers or less, or about 10 micrometers or less. Particles of such sizes can be prepared using a comminuting method known to those skilled in the art, such as spiral jet milling, fluid bed jet milling, supercritical fluid processing to form nanoparticles, high pressure homogenization, or spray drying.

Capsules, blisters, and cartridges for use in an inhaler or insufflator can be formulated to contain a powder mix of the pharmaceutical composition provided herein; a suitable powder base, such as lactose or starch; and a performance modifier, such as l-leucine, mannitol, or magnesium stearate. The lactose may be anhydrous or in the form of the monohydrate. Other suitable excipients or carriers include, but are not limited to, dextran, glucose, maltose, sorbitol, xylitol, fructose, sucrose, and trehalose. The pharmaceutical composition provided herein for inhaled/intranasal administration can further comprise a suitable flavor, such as menthol and levomenthol; and/or sweeteners, such as saccharin and saccharin sodium.

The pharmaceutical composition provided herein for topical administration can be formulated to be immediate release or modified release, including delayed-, sustained-, pulsed-, controlled-, targeted, and programmed release.

### D. Modified Release

The pharmaceutical composition provided herein can be formulated as a modified release dosage form. As used herein, the term “modified release” refers to a dosage form in which the rate or place of release of an active ingredient(s) is different from that of an immediate dosage form when administered by the same route. Modified release dosage forms include, but are not limited to, delayed-, extended-, prolonged-, sustained-, pulsatile-, controlled-, accelerated- and fast-, targeted-, programmed-release, and gastric retention dosage forms. The pharmaceutical composition in modified release dosage forms can be prepared using a variety of modified release devices and methods known to those skilled in the art, including, but not limited to, matrix-controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphism of the active ingredient(s).

### 1. Matrix Controlled Release Devices

The pharmaceutical composition provided herein in a modified release dosage form can be fabricated using a matrix-controlled release device known to those skilled in the art. See, e.g., Takada et al. in Encyclopedia of ControlledDrug Delivery, Mathiowitz Ed.; Wiley, 1999; Vol. 2.

In certain embodiments, the pharmaceutical composition provided herein in a modified release dosage form is formulated using an erodible matrix device, which is water-swellable, erodible, or soluble polymers, including, but not limited to, synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum Ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; cellulosics, such as ethyl cellulose (EC), methylethyl cellulose (MEC), carboxymethyl cellulose (CMC), CMEC, hydroxyethyl cellulose (HiEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethyl hydroxyethyl cellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT®); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; and other acrylic acid derivatives, such as homopolymers and copolymers of butylmethacrylate, methyl methacrylate, ethyl methacrylate, ethylacrylate, (2-dimethylaminoethyl)methacrylate, and (trimethylaminoethyl)methacrylate chloride.

In certain embodiments, the pharmaceutical composition provided herein is formulated with a non-erodible matrix device. The active ingredient(s) is dissolved or dispersed in an inert matrix and is released primarily by diffusion through the inert matrix once administered. Materials suitable for use as a non-erodible matrix device include, but are not limited to, insoluble plastics, such as polyethylene, polypropylene, polyisoprene, polyisobutylene, polybutadiene, polymethylmethacrylate, polybutylmethacrylate, chlorinated polyethylene, polyvinylchloride, methyl acrylate-methyl methacrylate copolymers, ethylene-vinyl acetate copolymers, ethylene/propylene copolymers, ethylene/ethyl acrylate copolymers, vinyl chloride copolymers with vinyl acetate, vinylidene chloride, ethylene and propylene, ionomer polyethylene terephthalate, butyl rubbers, epichlorohydrin rubbers, ethylene/vinyl alcohol copolymer, ethylene/vinyl acetate/vinyl alcohol terpolymer, ethylene/vinyloxyethanol copolymer, polyvinyl chloride, plasticized nylon, plasticized polyethylene terephthalate, natural rubber, silicone rubbers, polydimethylsiloxanes, and silicone carbonate copolymers; hydrophilic polymers, such as ethyl cellulose, cellulose acetate, crospovidone, and cross-linked partially hydrolyzed polyvinyl acetate; and fatty compounds, such as carnauba wax, microcrystalline wax, and triglycerides.

In a matrix-controlled release system, the desired release kinetics can be controlled, for example, via the polymer type employed, the polymer viscosity, the particle sizes of the polymer and/or the active ingredient(s), the ratio of the active ingredient(s) versus the polymer, and other excipients or carriers in the compositions.

The pharmaceutical composition provided herein in a modified release dosage form can be prepared by methods known to those skilled in the art, including direct compression, dry or wet granulation followed by compression, and melt-granulation followed by compression.

### 2. Osmotic Controlled Release Devices

The pharmaceutical composition provided herein in a modified release dosage form can be fabricated using an osmotic controlled release device, including, but not limited to, one-chamber system, two-chamber system, asymmetric membrane technology (AMT), and extruding core system (ECS). In general, such devices have at least two components: (a) a core which contains an active ingredient; and (b) a semipermeable membrane with at least one delivery port, which encapsulates the core. The semipermeable membrane controls the influx of water to the core from an aqueous environment of use so as to cause drug release by extrusion through the delivery port(s).

In addition to the active ingredient(s), the core of the osmotic device optionally includes an osmotic agent, which creates a driving force for transport of water from the environment of use into the core of the device. One class of osmotic agents is water-swellable hydrophilic polymers, which are also referred to as “osmopolymers” and “hydrogels.” Suitable water-swellable hydrophilic polymers as osmotic agents include, but are not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP), crosslinked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HiEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

The other class of osmotic agents is osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol; organic acids, such as ascorbic acid, benzoic acid, fumaric acid, citric acid, maleic acid, sebacic acid, sorbic acid, adipic acid, edetic acid, glutamic acid, p-toluenesulfonic acid, succinic acid, and tartaric acid; urea; and mixtures thereof.

Osmotic agents of different dissolution rates can be employed to influence how rapidly the active ingredient(s) is initially delivered from the dosage form. For example, amorphous sugars, such as MANNOGEM™ EZ can be used to provide faster delivery during the first couple of hours to promptly produce the desired therapeutic effect, and gradually and continually release of the remaining amount to maintain the desired level of therapeutic or prophylactic effect over an extended period of time. In this case, the active ingredient(s) is released at such a rate to replace the amount of the active ingredient metabolized and excreted.

The core can also include a wide variety of other excipients and carriers as described herein to enhance the performance of the dosage form or to promote stability or processing.

Materials useful in forming the semipermeable membrane include various grades of acrylics, vinyls, ethers, polyamides, polyesters, and cellulosic derivatives that are water-permeable and water-insoluble at physiologically relevant pHs or are susceptible to being rendered water-insoluble by chemical alteration, such as crosslinking. Examples of suitable polymers useful in forming the coating, include plasticized, unplasticized, and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CAp-toluene sulfonate, agar acetate, amylose triacetate, beta glucan acetate, beta glucan triacetate, acetaldehyde dimethyl acetate, triacetate of locust bean gum, hydroxylated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

Semipermeable membrane can also be a hydrophobic microporous membrane, wherein the pores are substantially filled with a gas and are not wetted by the aqueous medium but are permeable to water vapor, as disclosed in U.S. Pat. No. 5,798,119. Such hydrophobic but water-vapor permeable membrane are typically composed of hydrophobic polymers such as polyalkenes, polyethylene, polypropylene, polytetrafluoroethylene, polyacrylic acid derivatives, polyethers, polysulfones, polyethersulfones, polystyrenes, polyvinyl halides, polyvinylidene fluoride, polyvinyl esters and ethers, natural waxes, and synthetic waxes.

The delivery port(s) on the semipermeable membrane can be formed post-coating by mechanical or laser drilling. Delivery port(s) can also be formed in situ by erosion of a plug of water-soluble material or by rupture of a thinner portion of the membrane over an indentation in the core. In addition, delivery ports can be formed during coating process, as in the case of asymmetric membrane coatings of the type disclosed in U.S. Pat. Nos. 5,612,059 and 5,698,220.

The total amount of the active ingredient(s) released and the release rate can substantially by modulated via the thickness and porosity of the semipermeable membrane, the composition of the core, and the number, size, and position of the delivery ports.

The pharmaceutical composition in an osmotic controlled-release dosage form can further comprise additional conventional excipients or carriers as described herein to promote performance or processing of the formulation.

The osmotic controlled-release dosage forms can be prepared according to conventional methods and techniques known to those skilled in the art. See, e.g., Remington: The Science and Practice of Pharmacy, supra; Santus and Baker, J Controlled Release, 1995, 35, 1-21; Verma et al., Drug Dev. Ind. Pharm., 2000, 26, 695-708; Verma et al., J. Controlled Release, 2002, 79, 7-27.

In certain embodiments, the pharmaceutical composition provided herein is formulated as an AMT controlled-release dosage form, which comprises an asymmetric osmotic membrane that coats a core comprising the active ingredient(s) and other pharmaceutically acceptable excipients or carriers. See, e.g., U.S. Pat. No. 5,612,059 and WO 2002/17918. The AMT controlled-release dosage forms can be prepared according to conventional methods and techniques known to those skilled in the art, including direct compression, dry granulation, wet granulation, and a dip-coating method.

In certain embodiments, the pharmaceutical composition provided herein is formulated as an ESC controlled-release dosage form, which comprises an osmotic membrane that coats a core comprising the active ingredient(s), a hydroxyethyl cellulose, and other pharmaceutically acceptable excipients or carriers.

### 3. Multiparticulate Controlled Release Devices

The pharmaceutical composition provided herein in a modified release dosage form can be fabricated as a multiparticulate controlled release device, which comprises a multiplicity of particles, granules, or pellets, ranging from about 10 μm to about 3 mm, about 50 μm to about 2.5 mm, or from about 100 μm to about 1 mm in diameter. Such multiparticulates can be made by the processes known to those skilled in the art, including wet- and dry-granulation, extrusion/spheronization, roller-compaction, melt-congealing, and by spray-coating seed cores. See, e.g., Multiparticulate Oral Drug Delivery; Ghebre-Sellassie Eds.; Drugs and the Pharmaceutical Sciences 65; CRC Press: 1994; and Pharmaceutical Palletization Technology; Ghebre-Sellassie Eds.; Drugs and the Pharmaceutical Sciences 37; CRC Press: 1989.

Other excipients or carriers as described herein can be blended with the pharmaceutical composition to aid in processing and forming the multiparticulates. The resulting particles can themselves constitute the multiparticulate device or can be coated by various film-forming materials, such as enteric polymers, water-swellable, and water-soluble polymers. The multiparticulates can be further processed as a capsule or a tablet.

### 4. Targeted Delivery

The pharmaceutical composition provided herein can also be formulated to be targeted to a particular tissue, receptor, or other area of the body of the subject to be treated, including liposome-, resealed erythrocyte-, and antibody-based delivery systems. Examples include, but are not limited to, those disclosed in U.S. Pat. Nos. 6,316,652; 6,274,552; 6,271,359; 6,253,872; 6,139,865; 6,131,570; 6,120,751; 6,071,495; 6,060,082; 6,048,736; 6,039,975; 6,004,534; 5,985,307; 5,972,366; 5,900,252; 5,840,674; 5,759,542; and 5,709,874.

### Methods of Use

In one embodiment, provided herein is a method of treating, preventing, or alleviating one or more symptoms of a parasitic disease in a subject, comprising administering to the subject a therapeutically effective amount of a compound provided herein, e.g., a compound of Formula (A-I), (A-II), or (I), or an enantiomer, a mixture of enantiomers, or a mixture of diastereomers thereof, or a pharmaceutically acceptable salt, solvate, hydrate, or prodrug thereof.

In another embodiment, provided herein is a method of treating, preventing, or alleviating one or more symptoms of Chagas disease in a subject, comprising administering to the subject a therapeutically effective amount of a compound provided herein, e.g., a compound of Formula (A-I), (A-II), or (I), or an enantiomer, a mixture of enantiomers, or a mixture of diastereomers thereof, or a pharmaceutically acceptable salt, solvate, hydrate, or prodrug thereof.

In certain embodiments, the subject to be treated with a method provided herein has not been treated with an anti-parasite therapy for the parasitic disease before. In certain embodiments, the subject to be treated with a method provided herein has been treated with an anti-parasite therapy for the parasitic disease before.

In certain embodiments, the compound used in a method provided herein is delivered as a single dose such as, e.g., as a single bolus injection, or as a single oral tablet or pill. In certain embodiments, the compound used in a method provided herein is administered over time, such as, e.g., continuous infusion over time or divided bolus doses over time.

In certain embodiments, the compound used in a method provided herein is administered once daily (QD), or divided into multiple daily doses such as twice daily (BID), three times daily (TID), four times daily (QID), five times daily, six times daily, seven times daily, eight times daily, nine times daily, or ten times daily.

In certain embodiments, the compound used in a method provided herein is administered from about 1 to about 20 times a day, from about 1 to about 15 times a day, from about 1 to about 10 times a day, or from about 1 to about 5 times a day. In certain embodiments, the compound used in a method provided herein is administered every 1 to 10 hour(s), every 2 to 8 hours, every 3 to 7 hours, every 4 to 6 hours, or every 5 to 6 hours. In certain embodiments, the compound used in a method provided herein is administered every hour, every 2 hours, every 3 hours, every 4 hours, every 5 hours, every 6 hours, every 7 hours, every 8 hours, every 9 hours, or every 10 hours. In certain embodiments, the compound used in a method provided herein is administered once a day. In certain embodiments, the compound used in a method provided herein is administered 5 times a day. In certain embodiments, the compound used in a method provided herein is administered 10 times a day. In certain embodiments, the compound used in a method provided herein is administered every 4, 5, or 6 hours.

In certain embodiments, the compound used in a method provided herein is administered to the subject in a therapeutically effective amount ranging from about 0.01 to about 50 mg/kg/day, from about 0.015 to about 50 mg/kg/day, from about 0.05 to about 40 mg/kg/day, from about 0.2 to about 30 mg/kg/day, or from about 10 to about 30 mg/kg/day. In certain embodiments, the compound used in a method provided herein is administered to the subject in a therapeutically effective of about 0.015 mg/kg/day, about 5 mg/kg/day, about 25 mg/kg/day, or about 30 mg/kg/day.

The administered dose of the compound used in a method provided herein can also be expressed in a unit other than the unit “mg/kg/day” or “g/kg/day.” For example, a dose for parenteral administration can be expressed as mg/m²/day. One of ordinary skill in the art would readily know how to convert a dose from mg/kg/day to mg/m²/day, given either the height or weight of a subject or both. For example, a dose of 1 mg/kg/day for a 65 kg human approximately equals to 38 mg/m²/day.

In certain embodiments, the compound used in a method provided herein is administered to the subject in an amount ranging from about 0.2 mg to about 300 mg every four hours. In certain embodiments, the compound used in a method provided herein is administered to the subject in an amount ranging from about 3 mg to about 30 mg a day. In certain embodiments, the compound used in a method provided herein is administered to the subject in an amount ranging from about 3 mg to about 100 mg a day. In certain embodiments, the compound used in a method provided herein is administered to the subject in an amount of greater than about 3 mg a day. In certain embodiments, the compound used in a method provided herein is administered to the subject in an amount up to about 100 mg a day. In certain embodiments, the compound used in a method provided herein is administered to the subject in an amount up to about 150 mg a day. In certain embodiments, the compound used in a method provided herein is administered to the subject in an amount up to about 200 mg a day. In certain embodiments, the compound used in a method provided herein is administered to the subject in an amount up to about 300 mg a day. In certain embodiments, the compound used in a method provided herein is administered to the subject in an amount up to about 400 mg a day. In certain embodiments, the compound used in a method provided herein is administered to the subject in an amount up to about 500 mg a day. In certain embodiments, the compound used in a method provided herein is administered to the subject in an amount up to about 600 mg a day. In certain embodiments, the compound used in a method provided herein is administered to the subject in an amount up to about 700 mg a day. In certain embodiments, the compound used in a method provided herein is administered to the subject in an amount up to about 800 mg a day. In certain embodiments, the compound used in a method provided herein is administered to the subject in an amount up to about 900 mg a day. In certain embodiments, the compound used in a method provided herein is administered to the subject in an amount up to about 1,000 mg a day. In certain embodiments, the compound used in a method provided herein is administered to the subject in an amount up to about 2,000 mg a day.

Depending on the condition of the parasitic disease to be treated and the subject's condition, the compound used in a method provided herein can be administered by oral, parenteral (e.g., intramuscular, intraperitoneal, intravenous, CIV, intracisternal injection or infusion, subcutaneous injection, or implant), inhalation, nasal, vaginal, rectal, sublingual, or topical (e.g., transdermal or local) route of administration. In certain embodiments, the compound used in a method provided herein is administered independently by oral, parenteral, intravenous, or topical route of administration. The compound used in a method provided herein can be formulated in suitable dosage unit with one or more pharmaceutically acceptable excipients appropriate for each route of administration.

In one embodiment, the compound used in a method provided herein is administered orally. In another embodiment, the compound used in a method provided herein is administered parenterally. In yet another embodiment, the compound used in a method provided herein is administered intravenously. In still another embodiment, the compound used in a method provided herein is administered topically.

In certain embodiments, compound used in a method provided herein is administered to the subject after mealtime. In certain embodiments, compound used in a method provided herein is administered to the subject with a meal.

In certain embodiments, the subject is a mammal. In certain embodiments, the mammal is a human. In certain embodiments, the subject is one of livestock. In certain embodiments, the subject is a domesticated animal.

In certain embodiments, a method provided herein further comprises administering an additional therapeutic agent or therapy that is useful in treating, preventing, or ameliorating one or more symptoms of a parasitic disease. Effective dosages of the additional therapeutic agent can be administered together with, alternatively to, or sequentially to the administration of a compound provided herein. The dosages given will depend on absorption, inactivation, and excretion rates of the therapeutic agents as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the therapeutic agents. In certain embodiments, the additional therapeutic agent is benznidazole or nifurtimox.

In another embodiment, provided herein is a method of inhibiting the growth of a parasite, comprising contacting the parasite with an effective amount of a compound provided herein, e.g., a compound of Formula (A-I), (A-II), or (I), or an enantiomer, a mixture of enantiomers, or a mixture of diastereomers thereof, or a pharmaceutically acceptable salt, solvate, hydrate, or prodrug thereof.

In certain embodiments, the parasite is Plasmodium, Trypanosoma, Entamoeba, Giardia, Leishmania, Toxoplasma, or Schistosoma. In certain embodiments, the parasite is Plasmodium. In certain embodiments, the parasite is Trypanosoma. In certain embodiments, the parasite is Entamoeba. In certain embodiments, the parasite is Giardia. In certain embodiments, the parasite is Leishmania. In certain embodiments, the parasite is Toxoplasma. In certain embodiments, the parasite is Schistosoma. In certain embodiments, the parasite is Plasmodium falciparum, Plasmodium yoelii, Plasmodium berghei, Trypanosoma brucei, Trypanosoma cruzi, Entamoeba histolytica, Giardia lamblia, Leishmania major, Leishmania tropica, Leishmania aethiopica, Leishmania mexicana, Leishmania braziliensis, Leishmania donovani, Toxoplasma gondii, or Schistosoma mansonii. In certain embodiments, the parasite is Trypanosoma brucei. In certain embodiments, the parasite is Trypanosoma cruzi.

In certain embodiments, provided herein are kits which, when used by the medical practitioner, can simplify the administration of appropriate amounts of active ingredients to a subject. In certain embodiments, the kit provided herein includes containers and dosage forms of the active ingredients provided herein.

Kits provided herein can further include devices that are used to administer the active ingredients. Examples of such devices include, but are not limited to, syringes, needle-less injectors drip bags, patches, and inhalers. The kits provided herein can also include condoms for administration of the active ingredients.

Kits provided herein can further include pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: aqueous vehicles, including, but not limited to, Water for Injection USP, Sodium Chloride Injection, Ringer's Injection, Dextrose Injection, Dextrose and Sodium Chloride Injection, and Lactated Ringer's Injection; water-miscible vehicles, including, but not limited to, ethyl alcohol, polyethylene glycol, and polypropylene glycol; and non-aqueous vehicles, including, but not limited to, corn oil, cottonseed oil, peanut oil, sesame oil, ethyl oleate, isopropyl myristate, and benzyl benzoate.

The disclosure will be further understood by the following non-limiting examples.

### EXAMPLES

As used herein, the symbols and conventions used in these processes, schemes and examples, regardless of whether a particular abbreviation is specifically defined, are consistent with those used in the contemporary scientific literature, for example, the Journal of the American Chemical Society, the Journal of Medicinal Chemistry, or the Journal of Biological Chemistry. Specifically, but without limitation, the following abbreviations may be used in the examples and throughout the specification: g (grams); mg (milligrams); mL (milliliters); μL (microliters); mM (millimolar); μM (micromolar); mmol (millimoles); min (minute or minutes); h (hour or hours); ACN (acetonitrile); Co(salen) (N,N′-bis(salicylidene)ethylenediamino cobalt(II)); DCM (dichloromethane); DMF (N,N-dimethylacetamide); DMSO (dimthyl-sulfoxide); EtOAc (ethyl acetate); EtOH (ethanol); Fremy's salt (potassium nitrosodisulfonate); Grubbs catalyst 2nd generation (1,3-bis(2,4,6-trimethylphenyl)-2-imidazolidinylidene)dichloro-(phenylmethylene)(tricyclohexylphosphine)ruthenium); MeOH (methanol); NaSEt (sodium ethanethiolate); NMP (N-methyl-2-pyrrolidone); OAc (acetate); PE (petroleum ether); RT (room temperature); LCMS (liquid chromatography-mass spectrometry); NMR (nuclear magnetic resonance); and TLC (thin-layer chromatography).

For all of the following examples, standard work-up and purification methods known to those skilled in the art can be utilized. Unless otherwise indicated, all temperatures are expressed in ° C. (degrees Centigrade). All reactions are conducted at room temperature unless otherwise specified. Synthetic methodologies illustrated herein are intended to exemplify the applicable chemistry through the use of specific examples and are not indicative of the scope of the disclosure.

### Example 1

### Preparation of 6-methylbenzo[b][1,4]dioxine-5,8-dione A1 and sodium 6-methyl-5,8-dioxo-5,6,7,8-tetrahydrobenzo[b][1,4]dioxine-6-sulfonate A4

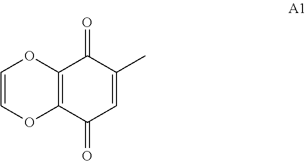

Compounds A1 and A4 were prepared as shown in Scheme 1.

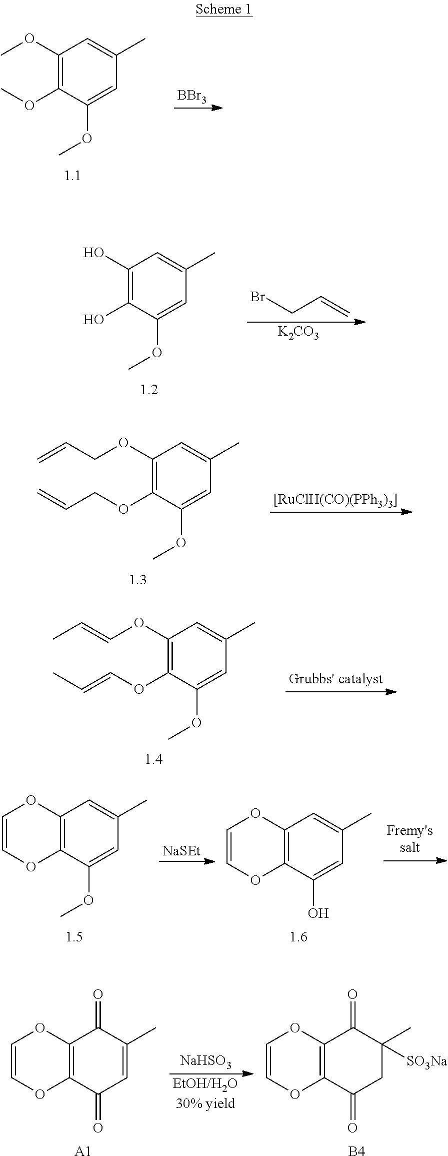

3-Methoxy-5-methylbenzene-1,2-diol 1.2. To a solution of compound 1.1 (30 g, 165 mmol) in DCM (250 mL) at −20° C. under N₂ was added 1M BBr₃ in DCM (181 mL, 181 mmol) dropwise. After stirred at −20° C. for 30 min, the reaction mixture was poured into water (300 mL), extracted with DCM (3×150 mL), dried over anhydrous Na₂SO₄, and concentrated under reduced pressure to afford compound 1.2 (19.2 g) in 75% yield.

1,2-Bis(allyloxy)-3-methoxy-5-methylbenzene 1.3. To a solution of compound 1.2 (19.2 g, 125 mmol) in ACN (300 mL) were added K₂CO₃ (69 g, 499 mmol, 4 eq.) and allyl bromide (60.3 g, 499 mmol). After refluxed for 16 h, the reaction mixture was cooled to room temperature, diluted with water (300 mL), and extracted with EtOAc (3×300 mL). The combined organic layers were washed with brine, dried over anhydrous Na₂SO₄, and concentrated under reduced pressure to yield a crude product, which was purified by silica-gel column chromatography eluting with EtOAc in PE to afford compound 1.3 (26.3 g) in 89% yield.

1-Methoxy-5-methyl-2,3-bis(((E)-prop-1-en-1-yl)oxy)benzene 1.4. To a solution of compound 1.3 (37 g, 158 mmol) in toluene (250 mL) was added RuClH(CO)(PPh₃)₃ (7.5 g, 7.9 mmol). After refluxed for 16 h under N₂, the reaction mixture was concentrated under reduced pressure to yield a crude product, which was purified by silica-gel column chromatography eluting with EtOAc in PE to afford compound 1.4 (34.8 g) in 94% yield.

5-Methoxy-7-methylbenzo[b][1,4]dioxine 1.5. To a solution of compound 1.4 (34.8 g, 148 mmol) in toluene (250 mL) was added Grubbs' catalyst 2nd generation (5 g, 5.9 mmol). After refluxed for 16 h under N₂, the reaction mixture was concentrated under reduced pressure to yield a crude product, which was purified by silica-gel column chromatography eluting with EtOAc in PE to afford compound 1.5 (20.1 g) in 76% yield.

7-Methylbenzo[b][1,4]dioxin-5-ol 1.6. To a solution of compound 1.5 (12 g, 67.4 mmol) in DMF (350 mL) was added NaSEt (23 g, 270 mmol) at room temperature. After stirred at 110° C. for 16 h under N₂, the reaction mixture was cooled to room temperature, diluted with water (1 L), and extracted with EtOAc (500 mL×2). The combined organic layers were washed with saturated sodium bicarbonate and then brine, dried over anhydrous Na₂SO₄, and concentrated under reduced pressure to yield a crude product, which was purified by silica-gel column chromatography eluting with EtOAc in PE to afford compound 1.6 (11.3 g) in a quantitative yield.

6-Methylbenzo[b][1,4]dioxine-5,8-dione A1. To a solution of Fremy′ salt (potassium nitrosodisulfonate) (8.1 g, 30.4 mmol) and sodium hydrogen phosphate (6.8 g, 48.6 mmol) in ACN/water (15 mL/250 mL) was added a solution of compound 1.6 (5 g, 30.4 mmol) in ACN (30 mL) dropwise at 0° C. After stirred at room temperature for 2 h under N₂, the reaction mixture was extracted with EtOAc (100 mL×2). The combined organic layers were washed with brine, dried over anhydrous Na₂SO₄, and concentrated under reduced pressure to yield a crude product, which was purified by silica-gel column chromatography eluting with EtOAc in PE to afford compound A1 (2.3 g) in 43% yield. ¹H NMR (400 MHz, CD₃OD): δ 6.45 (s, 1H), 6.07-6.05 (m, 2H), 1.98 (s, 3H); LCMS (ESI) m/z: 179.0 [M+H]⁺.

Sodium 6-methyl-5,8-dioxo-5,6,7,8-tetrahydrobenzo[b][1,4]dioxine-6-sulfonate A4. Compound A4 was prepared according to the procedures as described in U.S. Pat. No. 3,657,286 in 66% yield. ¹H NMR (400 MHz, DMSO-d₆): δ 6.22-6.19 (q, 2H), 2.86-2.68 (q, 2H), 1.25 (s, 3H); LCMS (ESI) m/z: 258.9 [M−H]⁻.

### Example 2

### Preparation of 7-methyl-3,4-dihydro-2H-chromene-5,8-dione B1 and sodium 6-methyl-5,8-dioxo-2,3,5,6,7,8-hexahydrobenzo[b][1,4]dioxine-6-sulfonate B4

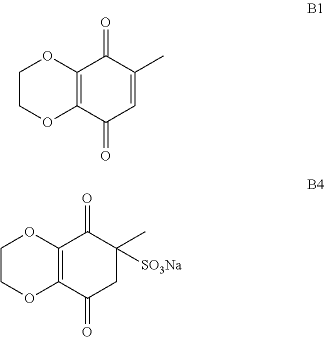

Compounds B1 and B4 were prepared as shown in Scheme 2.

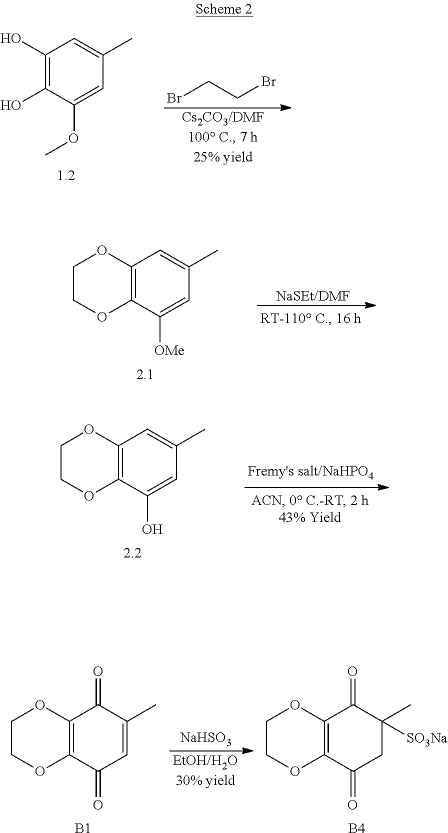

7-Methyl-3,4-dihydro-2H-chromene-5,8-dione B1. Compound B1 was prepared similarly according to the procedures described in Example 1 for compound A1. ¹H NMR (400 MHz, CDCl₃): δ 6.39-6.38 (q, 1H), 4.31 (s, 4H), 2.03 (s, 3H).

Sodium 6-methyl-5,8-dioxo-2,3,5,6,7,8-hexahydrobenzo[b][1,4]dioxine-6-sulfonate B4. Compound B4 was prepared similarly according to the procedures described in Example 1 for compound A4. ¹H NMR (400 MHz, DMSO-d₆): δ 4.35-4.30 (m, 2H), 3.97-3.94 (m, 2H), 2.92-2.88 (d, 1H), 2.72-2.68 (d, 1H), 1.27 (s, 3H); LCMS (ESI) m/z: 260.8 [M−H]⁻.

### Example 3

### Preparation of 7-methyl-3,4-dihydro-2H-chromene-5,8-dione C1

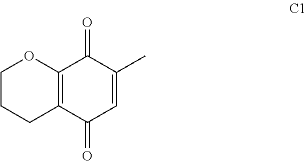

Compound C1 was prepared as shown in Scheme 3.

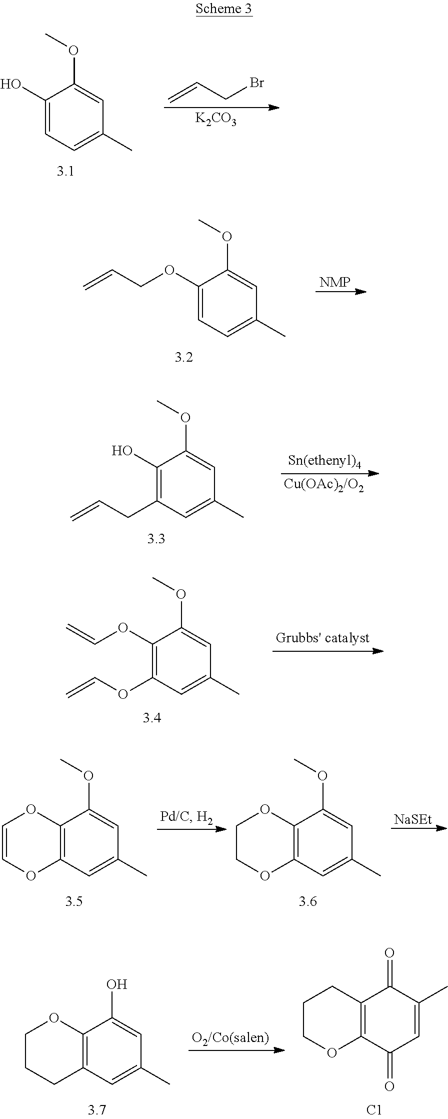

1-(Allyloxy)-2-methoxy-4-methylbenzene 3.2. To a solution of 2-methoxy-4-methylphenol 3.1 (90 g, 651 mmol) in acetone (900 mL) were added 3-bromoprop-1-ene (94.6 g, 782) and K₂CO₃ (108 g, 782 mmol). After stirred at 60° C. under N₂ for 16 h, the reaction mixture was filtered and washed with acetone. The filtrate was concentrated under reduced pressure to yield a crude product, which was purified by silica-gel column chromatography eluting with EtOAc in PE to afford compound 3.2 (110 g) in 95% yield.

2-Allyl-6-methoxy-4-methylphenol 3.3. A solution of compound 3.2 (50 g, 281 mmol) in NMP (500 mL) was stirred at 220° C. for 6 h. After cooled to room temperature, the reaction mixture was diluted with EtOAc (600 mL), washed with water and brine, dried over anhydrous Na₂SO₄, filtered, and concentrated under reduced pressure to yield a crude product, which was purified by silica-gel column chromatography eluting with EtOAc in PE to afford compound 3.3 (45.6 g) in 91% yield.

1-Allyl-3-methoxy-5-methyl-2-(vinyloxy)benzene 3.4. To a solution of compound 3.3 (16.5 g, 92.6 mmol) in dry ACN (170 mL) were added tetravinylstannane (25.2 g, 111 mmol) and Cu(OAc)₂ (20.0 g, 111 mmol). After stirred at room temperature for 16 h under oxygen atmosphere, the reaction mixture was diluted with EtOAc (300 mL), washed with brine, dried over anhydrous Na₂SO₄, filtered, and concentrated under reduced pressure to yield a crude product, which was purified by silica-gel column chromatography eluting with EtOAc in PE to afford compound 3.4 (16.2 g) in 86% yield.

8-Methoxy-6-methyl-4H-chromene 3.5. To a solution of compound 3.4 (16.2 g, 79.3 mmol) in toluene (160 mL) was added Grubbs' catalyst 2nd generation (2.7 g, 3.2 mmol). After stirred at 60° C. under N₂ for 1 h, the reaction mixture was concentrated under reduced pressure to afford compound 3.5 (16.2 g), which was directly used for next step reaction without further purification.

8-Methoxy-6-methylchromane 3.6. A solution of compound 3.5 (16.2 g, 91.9 mmol) in MeOH (160 mL) was added Pd/C (10%, 3.24 g). After stirred at room temperature for 16 h under hydrogen atmosphere, the reaction mixture was filtered and concentrated under reduced pressure to afford compound 3.6 (16 g), which was directly used for next step reaction without further purification.

6-Methylchroman-8-ol 3.7. To a solution of compound 3.6 (16 g, 89.8 mmol) in DCM (160 mL) was added 1M BBr₃ in DCM (89.8 mL, 89.8 mmol) at 0° C. After stirred at 0° C. for 2 h, the reaction mixture was diluted with water (100 mL) and extracted with DCM (200 mL×3). The combined organic layers were washed with brine, dried over anhydrous Na₂SO₄, filtered, and concentrated under reduced pressure to yield a crude product, which was purified by silica-gel column chromatography eluting with EtOAc in PE to afford compound 3.7 (14 g) in 95% yield.

7-methyl-3,4-dihydro-2H-chromene-5,8-dione C1. To a solution of compound 3.7 (14 g, 85.3 mmol) in DMF (140 mL) was added Co(salen) (27.7 g, 85.3 mmol). After stirred at room temperature for 16 h under oxygen atmosphere, the reaction mixture was diluted with water (100 mL) and extracted with EtOAc (100 mL×3). The combined organic layers were washed with brine, dried over anhydrous Na₂SO₄, filtered, and concentrated under reduced pressure to yield a crude product, which was purified by silica-gel column chromatography eluting with EtOAc in PE to afford compound C1 (2.5 g) in 16% yield. ¹H NMR (400 MHz, CD₃OD): δ 6.47 (q, J=1.6 Hz, 1H), 4.40-3.88 (m, 2H), 2.38 (t, J=6.4 Hz, 2H), 1.99 (d, J=1.6 Hz, 3H), 1.97-1.88 (m, 2H); LCMS (ESI) m/z: 179.1 [M+H]⁺.

### Example 4

### Preparation of 6-methyl-3,4-dihydro-2H-chromene-5,8-dione D1

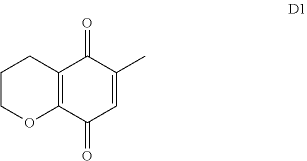

Compound D1 was prepared as shown in Scheme 4.

3-Hydroxy-2-methoxybenzaldehyde 4.2. To a mixture of compound 4.1 (60 g, 435 mmol) in DMF (600 mL) were added iodomethane (61.7 g, 435 mmol) and K₂CO₃ (120 g, 870 mmol). After stirred at room temperature under N₂ for 16 h, the reaction mixture was diluted with water (1 L) and extracted with EtOAc (800 mL×3). The combined organic layers were washed with brine (600 mL), dried over anhydrous Na₂SO₄, filtered and concentrated under reduced pressure to yield a crude product, which was purified by silica-gel column chromatography eluting with EtOAc in PE to afford compound 4.2 (43 g) in 65% yield.

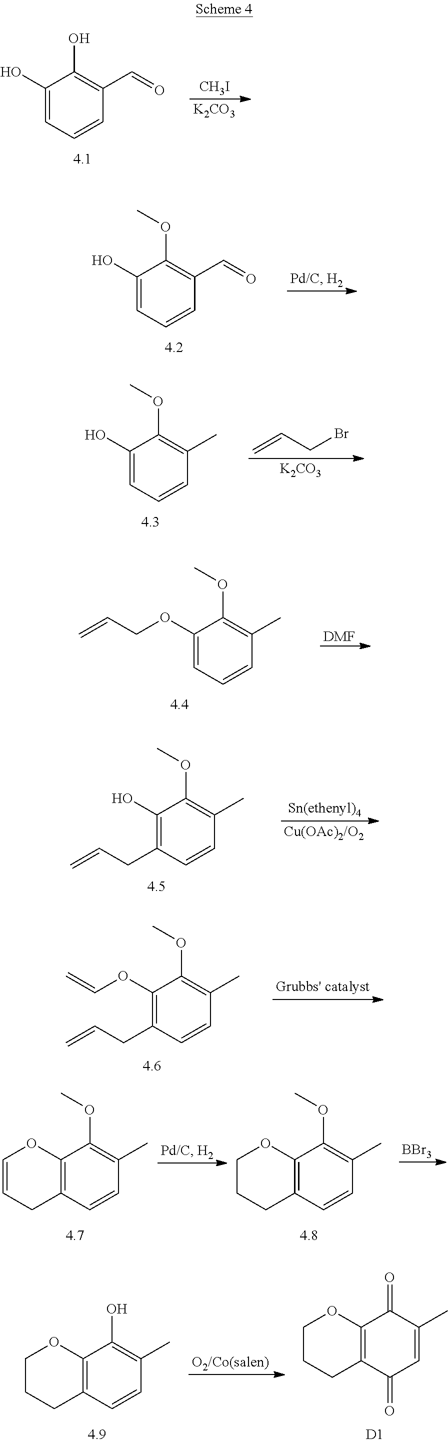

2-Methoxy-3-methylphenol 4.3. To a mixture of compound 4.2 (42 g, 276 mmol) in EtOH (400 mL) and AcOH (100 mL) was added Pd/C (10%, 4.2 g). The mixture was stirred at 60° C. for 48 h under H₂, the mixture was filtered through a pad of celite. The filtrate was concentrated under reduced pressure to yield a crude product, which was purified by silica-gel column chromatography eluting with EtOAc in PE to afford compound 4.3 (30 g) in 79% yield.

1-(Allyloxy)-2-methoxy-3-methylbenzene 4.4. To a mixture of compound 4.3 (30 g, 217 mmol) in acetone (300 mL) were added compound 4.3 (31.5 g, 261 mmol) and K₂CO₃ (60 g, 435 mmol). After stirred at 60° C. for 16 h under N₂, the reaction mixture was filtered and concentrated under reduced pressure to yield a crude product, which was purified by silica-gel column chromatography eluting with EtOAc in PE to afford compound 4.4 (24.3 g) in 63% yield.

6-Allyl-2-methoxy-3-methylphenol 4.5. A solution of compound 4.4 (24.3 g, 137 mmol) in NMP (150 mL) was stirred at 240° C. for 1 h under N₂. The reaction mixture was then cooled to room temperature, diluted with EtOAc (600 mL), washed with brine (300 mL×2), dried over anhydrous Na₂SO₄, filtered, and concentrated under reduced pressure to yield a crude product, which was purified by silica-gel column chromatography eluting with EtOAc in PE to afford compound 4.5 (14.3 g) in 59% yield.

1-Allyl-3-methoxy-4-methyl-2-(vinyloxy)benzene 4.6. To a mixture of compound 4.5 (16 g, 89.9 mmol) in ACN (200 mL) were added tetravinylstannane (26.6 g, 117 mmol) and copper acetate (32.5 g, 180 mmol). After stirred at room temperature for 16 h under oxygen atmosphere, the reaction mixture was filtered through a pad of celite. The filtrate was concentrated under reduced pressure to yield a crude product, which was purified by silica-gel column chromatography eluting with EtOAc in PE to afford compound 4.6 (10.3 g) in 56% yield.

8-Methoxy-7-methyl-4H-chromene 4.7. To a mixture of compound 4.6 (10.3 g, 50.5 mmol) in toluene (100 mL) was added Grubbs' catalyst 2nd generation (10%, 1 g). After stirred at 60° C. for 1 h under N₂, the reaction mixture was cooled to room temperature and concentrated under reduced pressure to afford compound 4.7 (13 g), which was directly used in the next step without further purification.

8-Methoxy-7-methylchromane 4.8. To a mixture of compound 4.7 (13 g) in MeOH (100 mL) was added Pd/C (10%, 1.3 g). After stirred at room temperature for 16 h under H₂, the reaction mixture was filtered through a pad of celite. The filtrate was concentrated under reduced pressure to yield a crude product, which was purified by silica-gel column chromatography eluting with EtOAc in PE to afford compound 4.8 (8.3 g).

7-Methylchroman-8-ol 4.9. To a mixture of compound 4.8 (8.3 g, 46.6 mmol) in DCM (90 mL) at 0° C. was dropwise added 1N BBr₃ in DCM (93.2 mL, 93.2 mmol). After stirred for 1 h under N₂, the reaction mixture was diluted with water (100 mL) and extracted with DCM (3×100 mL). The organic layers were washed with brine (200 mL), dried over anhydrous Na₂SO₄, filtered, and concentrated under reduced pressure to yield a crude product, which was purified by silica-gel column chromatography eluting with EtOAc in PE to afford compound 4.9 (7.5 g) in 99% yield.

6-Methyl-3,4-dihydro-2H-chromene-5,8-dione D1. To a mixture of compound 4.9 (8.1 g, 49.4 mmol) in DMF (80 mL) was added Co(salen) (16.1 g, 49.4 mmol). After stirred for 16 h under oxygen atmosphere, the reaction mixture was diluted with water (300 mL) and extracted with EtOAc (200 mL×3). The combined organic layers were washed with brine, dried over anhydrous Na₂SO₄, filtered, and concentrated under reduced pressure to yield a crude product, which was purified by silica-gel column chromatography eluting with EtOAc in PE to afford compound D1 (1.5 g) in 17% yield. ¹H NMR (400 MHz, CD₃OD): δ 6.47 (q, J=1.6 Hz, 1H), 4.28-4.19 (m, 2H), 2.41 (t, J=6.4 Hz, 2H), 2.02 (d, J=1.6 Hz, 3H), 1.97-1.90 (m, 2H); LCMS (ESI) m/z: 179.1 [M+H]⁺.

### Example B1

Antiparasitic Activity Against Trypanosoma cruzi

To evaluate antiparasitic activity against Trypanosoma cruzi trypomastigote, parasites were obtained from the supernatant of infected LLCMK2 cells. Trypomastigote (1×10⁷ parasites/mL) was resuspended in DMEM supplemented with 10% FBS. The parasites were incubated in the presence of a compound at 500, 200, 100, 50, 10, 5, 1, 0.5, and 0.25 μM in 96-well plates for 24 h at 37° C. with 5% CO₂. After incubation, the viability of the parasites was determined by examining mobility under an optical microscope. See, e.g., Brener, Rev. Inst. Med. Trop. São Paulo. 1962, 4, 389-96. Compound A1 was determined to have an EC₅₀ of 2.3±0.3 μM against Trypanosoma cruzi trypomastigote.

Similarly, compounds A2, A3, B1 to B3, C1 to C3, and D1 to D3 are evaluated for their antiparasitic activity against Trypanosoma cruzi trypomastigote.

The examples set forth above are provided to give those of ordinary skill in the art with a complete disclosure and description of how to make and use the claimed embodiments, and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

What is claimed is:

1. A compound of Formula (A-I): 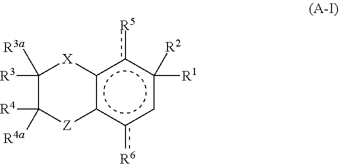
or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, hydrate, or prodrug thereof; wherein:
the symbol “ [symbol] ” represents a single bond or double bond;
X and Z are each independently O or C(R⁷R⁸);
R¹ is absent or —SO₃H;
R² is C₁₋₆ alkyl or C₁₋₆ heteroalkyl;
each R³, R⁴, R⁷, and R⁸ is independently (i) hydrogen, deuterium, cyano, or halo; or (ii) C₁₋₆ alkyl or C₁₋₆ heteroalkyl;
R⁵ and R⁶ are each independently —OH or ═O; and
R^{3a} and R^{4a} are each independently (i) hydrogen, deuterium, cyano, or halo; or (ii) C₁₋₆ alkyl or C₁₋₆ heteroalkyl; or R^{3a} and R^{4a} together with the carbon atoms to which they are attached form a double bond;
wherein each alkyl and heteroalkyl is optionally substituted with one or more, in one embodiment, one, two, three, or four, substituents Q, wherein each Q is independently selected from: (a) deuterium, cyano, halo, imino, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{a})OR^{d}, —OS(O)R^{a}, —OS(O)₂R^{a}, —OS(O)NR R^{c}, —OS(O)₂NR^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a};
wherein each Q^{a} is independently selected from: (a) deuterium, cyano, halo, nitro, imino, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OP(O)(OR^{f})OR^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NRC(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

2. The compound of claim 1, wherein X is O.

3. The compound of claim 1, wherein X is C(R⁷R⁸).

4. The compound of claim 1 or 3, wherein X is C(H₂).

5. The compound of any one of claims 1 to 4, wherein Z is O.

6. The compound of any one of claims 1 to 4, wherein Z is C(R⁷R⁸).

7. The compound of any one of claims 1 to 4 and 6, wherein Z is C(H₂).

8. The compound of claim 1, 2, or 5, wherein the compound is a compound of Formula (A-II): 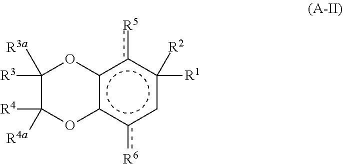
or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, hydrate, or prodrug thereof.

9. The compound of any one of claims 1, 2, 5, and 8, wherein the compound is a compound of Formula (I): 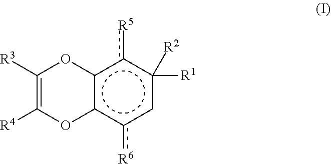
or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, hydrate, or prodrug thereof.

10. The compound of any one of claims 1, 2, 5, 8, and 9, wherein the compound is a compound of Formula (II): 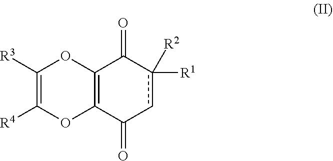
or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, hydrate, or prodrug thereof, wherein the symbol “ [symbol] ” represents a carbon-carbon single bond or double bond; and R¹ is (i) absent when the symbol “ [symbol] ” represents a carbon-carbon double bond or (ii) —SO₃H when the symbol “ [symbol] ” represents a carbon-carbon double bond.

11. The compound of any one of claims 1, 2, 5, and 8 to 10, wherein the compound is a compound of Formula (III): 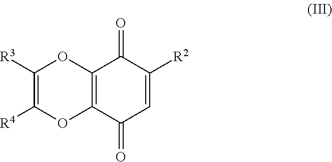
or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, hydrate, or prodrug thereof.

12. The compound of any one of claims 1, 2, 5, and 8 to 10, wherein the compound is a compound of Formula (IV): 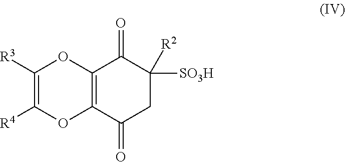
or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, hydrate, or prodrug thereof.

13. The compound of any one of claims 1, 2, 5, 8, and 9, wherein the compound is a compound of Formula (V): 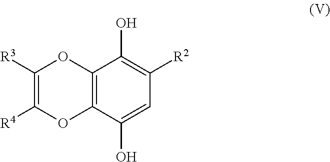
or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, hydrate, or prodrug thereof.

14. The compound of any one of claims 1, 2, 5, and 8, wherein the compound is a compound of Formula (IX): 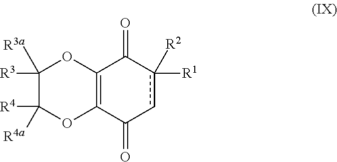
or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, hydrate, or prodrug thereof, wherein the symbol “ [symbol] ” represents a carbon-carbon single bond or double bond; and R¹ is (i) absent when the symbol “ [symbol] ” represents a carbon-carbon double bond or (ii) —SO₃H when the symbol “ [symbol] ” represents a carbon-carbon double bond.

15. The compound of any one of claims 1, 2, 5, 8, and 14, wherein the compound is a compound of Formula (X): 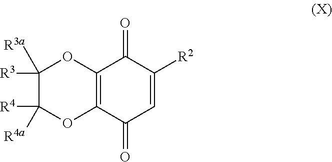
or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, hydrate, or prodrug thereof.

16. The compound of any one of claims 1, 2, 5, 8, and 14, wherein the compound is a compound of Formula (XI): 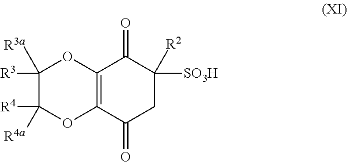
or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, hydrate, or prodrug thereof.

17. The compound of any one of claims 1, 2, 5, and 8, wherein the compound is a compound of Formula (XII): 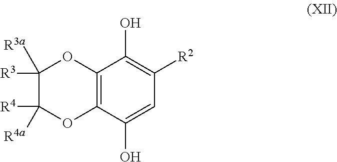
or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, hydrate, or prodrug thereof.

18. The compound of any one of claims 1, 2, and 6, wherein X is O and Z is C(R⁷R⁸).

19. The compound of any one of claims 1, 2, 6, and 18, wherein X is O and Z is C(H₂).

20. The compound of any one of claims 1, 18, and 19, wherein the compound is a compound of Formula (XVI): 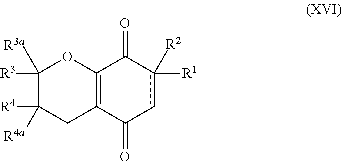
or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof, wherein the symbol “ [symbol] ” represents a carbon-carbon single bond or double bond; and R¹ is (i) absent when the symbol “ [symbol] ” represents a carbon-carbon double bond or (ii) —SO₃H when the symbol “ [symbol] ” represents a carbon-carbon double bond.

21. The compound of any one of claims 1, 18, and 19, wherein the compound is a compound of Formula (XVII): 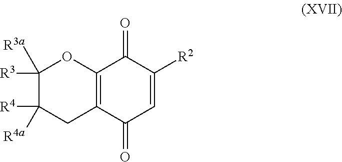
or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, hydrate, or prodrug thereof.

22. The compound of any one of claims 1, 18, and 19, wherein the compound is a compound of Formula (XVIII): 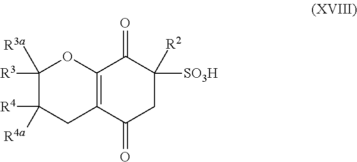
or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, hydrate, or prodrug thereof.

23. The compound of any one of claims 1, 18, and 19, wherein the compound is a compound of Formula (XIX): 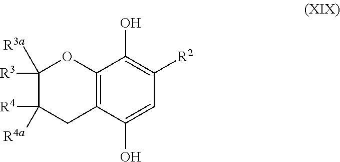
or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, hydrate, or prodrug thereof.

24. The compound of any one of claims 1, 3, and 5, wherein X is C(R⁷R⁸) and Z is O.

25. The compound of any one of claims 1, 3, 5, and 24, wherein X is C(H₂) and Z is O.

26. The compound of any one of claims 1, 24, and 25, wherein the compound is a compound of Formula (XXIII): 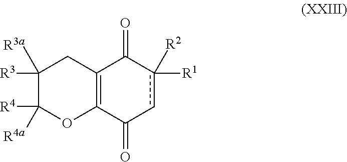
or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, hydrate, or prodrug thereof, wherein the symbol “ [symbol] ” represents a carbon-carbon single bond or double bond; and R¹ is (i) absent when the symbol “ [symbol] ” represents a carbon-carbon double bond or (ii) —SO₃H when the symbol “ [symbol] ” represents a carbon-carbon double bond.

27. The compound of any one of claims 1, 24, and 25, wherein the compound is a compound of Formula (XXIV): 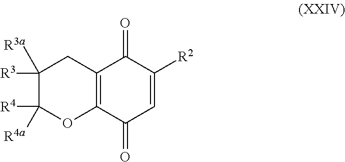
or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, hydrate, or prodrug thereof.

28. The compound of any one of claims 1, 24, and 25, wherein the compound is a compound of Formula (XXV): 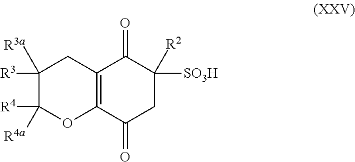
or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, hydrate, or prodrug thereof.

29. The compound of any one of claims 1, 24, and 25, wherein the compound is a compound of Formula (XXVI): 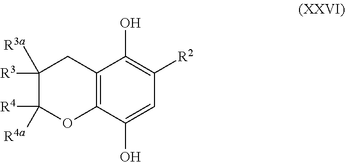
or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, hydrate, or prodrug thereof.

30. The compound of any one of claims 1 to 8 and 14 to 29, wherein R^{3a} is hydrogen or deuterium.

31. The compound of any one of claims 1 to 8 and 14 to 30, wherein R^{3a} is hydrogen.

32. The compound of any one of claims 1 to 8 and 14 to 31, wherein R^{4a} is hydrogen or deuterium.

33. The compound of any one of claims 1 to 8 and 14 to 32, wherein R^{4a} is hydrogen.

34. The compound of any one of claims 1 to 8 and 14 to 33, wherein R^{3a} and R^{4a} are each hydrogen.

35. The compound of any one of claims 1 to 34, wherein R² is C₁₋₆ alkyl, optionally substituted with one or more substituents Q.

36. The compound of any one of claims 1 to 35, wherein R² is methyl, ethyl, or propyl.

37. The compound of any one of claims 1 to 36, wherein R² is methyl.

38. The compound of any one of claims 1 to 37, wherein R³ is hydrogen or deuterium.

39. The compound of any one of claims 1 to 38, wherein R³ is hydrogen.

40. The compound of any one of claims 1 to 37, wherein R³ is cyano or halo.

41. The compound of any one of claims 1 to 37 and 40, wherein R³ is cyano, fluoro, chloro, or bromo.

42. The compound of any one of claims 1 to 37, wherein R³ is C₁₋₆ alkyl, optionally substituted with one or more substituents Q.

43. The compound of any one of claims 1 to 37 and 42, wherein R³ is methyl, ethyl, or propyl.

44. The compound of any one of claims 1 to 37, wherein R³ is C₁₋₆ heteroalkyl, optionally substituted with one or more substituents Q.

45. The compound of any one of claims 1 to 37 and 44, wherein R³ is hydroxylmethyl, 2-hydroxylethyl, or 2-methoxyethyl.

46. The compound of any one of claims 1 to 45, wherein R⁴ is hydrogen or deuterium.

47. The compound of any one of claims 1 to 46, wherein R⁴ is hydrogen.

48. The compound of any one of claims 1 to 45, wherein R⁴ is cyano or halo.

49. The compound of any one of claims 1 to 45 and 48, wherein R⁴ is cyano, fluoro, chloro, or bromo.

50. The compound of any one of claims 1 to 45, wherein R⁴ is C₁₋₆ alkyl, optionally substituted with one or more substituents Q.

51. The compound of any one of claims 1 to 45 and 50, wherein R⁴ is methyl, ethyl, or propyl.

52. The compound of any one of claims 1 to 45, wherein R⁴ is C₁₋₆ heteroalkyl, optionally substituted with one or more substituents Q.

53. The compound of any one of claims 1 to 45 and 52, wherein R⁴ is hydroxylmethyl, 2-hydroxylethyl, or 2-methoxyethyl.

54. The compound of any one of claims 1 to 39, 46, and 47, wherein R³ and R⁴ are each hydrogen.

55. The compound of claim 1, wherein the compound is:
6-methylbenzo[b][1,4]-dioxine-5,8-dione A1;
6-methyl-5,8-dioxo-5,6,7,8-tetrahydrobenzo[b][1,4]dioxine-6-sulfonic acid A2;
6-methylbenzo[b][1,4]dioxine-5,8-diol A3;
6-methyl-2,3-dihydrobenzo[b][1,4]dioxine-5,8-dione B1;
6-methyl-5,8-dioxo-2,3,5,6,7,8-hexahydrobenzo[b][1,4]dioxine-6-sulfonic acid B2;
6-methyl-2,3-dihydrobenzo[b][1,4]dioxine-5,8-diol B3;
7-methyl-3,4-dihydro-2H-chromene-5,8-dione C1;
7-methyl-5,8-dioxo-3,4,5,6,7,8-hexahydro-2H-chromene-7-sulfonic acid C2;
7-methylchromane-5,8-diol C3;
6-methyl-3,4-dihydro-2H-chromene-5,8-dione D1;
6-methyl-5,8-dioxo-3,4,5,6,7,8-hexahydro-2H-chromene-6-sulfonic acid D2; or
6-methylchromane-5,8-diol D3;
or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, hydrate, or prodrug thereof.

56. The compound of claim 1 or 55, wherein the compound is 6-methylbenzo[b][1,4]-dioxine-5,8-dione A1; or a tautomer, a mixture of two or more tautomers, or an isotopic variant thereof; or a pharmaceutically acceptable salt, solvate, hydrate, or prodrug thereof.

57. The compound of claim 1 or 55, wherein the compound is 6-methyl-5,8-dioxo-5,6,7,8-tetrahydrobenzo[b][1,4]dioxine-6-sulfonic acid A2; or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

58. The compound of claim 1, 55, or 57, wherein the compound is a pharmaceutically acceptable salt of 6-methyl-5,8-dioxo-5,6,7,8-tetrahydrobenzo[b][1,4]dioxine-6-sulfonic acid A2; or an enantiomer, a mixture of enantiomers, a diastereomer, a mixture of two or more diastereomers, a tautomer, a mixture of two or more tautomers, or an isotopic variant thereof; or
a pharmaceutically acceptable solvate, hydrate, or prodrug thereof.

59. The compound of any one of claims 1, 55, 57, and 58, wherein the compound is:
sodium 6-methyl-5,8-dioxo-5,6,7,8-tetrahydrobenzo[b][1,4]dioxine-6-sulfonate A4;
potassium 6-methyl-5,8-dioxo-5,6,7,8-tetrahydrobenzo[b][1,4]dioxine-6-sulfonate A5;
calcium 6-methyl-5,8-dioxo-5,6,7,8-tetrahydrobenzo[b][1,4]dioxine-6-sulfonate A6; or
magnesium 6-methyl-5,8-dioxo-5,6,7,8-tetrahydrobenzo[b][1,4]dioxine-6-sulfonate A7;
or an enantiomer, a mixture of enantiomers, a diastereomer, a mixture of two or more diastereomers, a tautomer, a mixture of two or more tautomers, or an isotopic variant thereof; or
a pharmaceutically acceptable solvate, hydrate, or prodrug thereof.

60. The compound of any one of claims 1, 55, 57, 58, and 59, wherein the compound is 6-methylbenzo[b][1,4]-dioxine-5,8-diol A3; or a tautomer, a mixture of two or more tautomers, or an isotopic variant thereof; or a pharmaceutically acceptable salt, solvate, hydrate, or prodrug thereof.

61. A pharmaceutical composition comprising the compound of any one of claims 1 to 60, or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, or hydrate thereof, and a pharmaceutically acceptable excipient.

62. The pharmaceutical composition of claim 61, wherein the composition is in single dosage form.

63. The pharmaceutical composition of claim 61 or 62, wherein the composition is in an oral, parenteral, or intravenous dosage form.

64. The pharmaceutical composition of claim 63, wherein the composition is formulated in an oral dosage form.

65. The pharmaceutical composition of claim 64, wherein the oral dosage form is a tablet or capsule.

66. A method of treating, preventing, or alleviating one or more symptoms of a parasitic disease in a subject, comprising administering to the subject in need thereof a therapeutically effective amount of a compound of any one of claims 1 to 60, or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, or hydrate thereof; or a pharmaceutical composition of any one of claims 61 to 65.

67. The method of claim 66, wherein the parasitic disease is Chagas disease.

68. The method of claim 66 or 67, further comprising administering an additional therapeutic agent.

69. The method of claim 68, wherein the additional therapeutic agent is an antiparasitic agent.

70. The method of claim 68 or 69, wherein the additional therapeutic agent is benznidazole or nifurtimox.

71. A method of inhibiting the growth of a parasite, comprising contacting the parasite with an effective amount of a compound of any one of claims 1 to 60, or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, or hydrate thereof; or a pharmaceutical composition of any one of claims 61 to 65.

72. The method of claim 71, wherein the parasite is Trypanosoma.

73. The method of claim 71 or 72, wherein the parasite is Trypanosoma cruzi.
